(19) United States
(12) Patent Application Publication
(10) Pub. No.: US 2015/0080234 A1
(43) Pub. Date: Mar. 19, 2015

(54) Cell Patterning

(71) Applicant: Massachusetts Institute of Technology, Cambridge, MA (US)

(72) Inventors: John Christopher Love, Somerville, MA (US); Ayca Yalcin Ozkumur, Jamaica Plain, MA (US); Brittany Anne Thomas, Cambrdige, MA (US)

(21) Appl. No.: 14/488,269

(22) Filed: Sep. 16, 2014

Related U.S. Application Data
(60) Provisional application No. 61/878,090, filed on Sep. 16, 2013.

(51) Int. Cl. G01N 33/487

(52) U.S. Cl. CPC G01N 33/48735
USPC 506/4; 506/14; 506/6; 506/26

(57) ABSTRACT

The present disclosure provides technologies for achieving cell patterning, as well as patterned cell arrays achieved with such technologies. Provided apparatus and methods are useful in a variety of contexts and provide particular advantages with respect to assessing living cell arrays and/or their responsiveness to stimuli, including identifying and/or characterizing complex cellular responses.

### CROSS-REFERENCE TO RELATED APPLICATION

This patent application claims priority to and the benefit of, U.S. provisional patent application Ser. No. 61/878,090, filed on Sep. 16, 2013, the entire contents of which are herein incorporated by reference.

### GOVERNMENT SUPPORT

This invention was made with government support under O.S.P Project No.: 6921222, awarded by the International AIDS Vaccine Initiative (IAVI). This invention was also made with government support under O.S.P Project No.: 6925064, awarded by the Rockefeller/Gates Collaboration for AIDS Vaccine Discovery (CAVD). This invention was also made with government support under O.S.P Project No.: 6920167, awarded by the Keck Foundation. The United States Government has certain rights in the invention.

### BACKGROUND

Control over the spatial patterning of cells on a surface is important for the study of cell behavior in a variety of simulated biological environments. A variety of methods have been developed for patterning cells on substrates. In general, such techniques typically rely on creating patterns of positive and negative adherence materials for cells to attach to or be repelled.

### SUMMARY

The present disclosure provides apparatus and methods useful for forming spatially registered patterns of cells. In particular, in many embodiments, the present disclosure utilizes a microwell device to generate a cell array that is spatially-registered relative to wells in the device. Provided apparatus and methods provide a variety of uses and advantages. Among other things, such provided apparatus and methods allow for correlation of cells to wells. In some embodiments, provided apparatus and methods permit production of cell arrays (e.g., arrays of living cells) that allows precise control over patterns and density of cells. In some embodiments, provided apparatus and methods contain or generate discrete cultures of desired cell types, in some embodiments some or all of such discrete cultures may be co-cultures. In some embodiments, provided technologies permit measurement of the effects of microenvironment on matterned cells. In some embodiments, provided apparatus and methods are useful for assessing functional responses of cells to environmental signal and/or stimuli of interest contained within wells. That is, in some embodiments, one or more wells may contain an agent or condition of interest (e.g., a biological or chemical agent, environmental condition, etc); impact of such agent or condition on patterned cells can readily be assessed in accordance with the present invention.

Among other things, the present invention identifies the source of a problem associated with various prior technologies for achieving cell patterning. For example, among other things, the present disclosure appreciates that reliance on complicated surface modifications and/or requirement for specialized equipment can reduce the utility and/or flexibility of many existing approaches to cell patterning. The present disclosure provides cell patterning technologies that, in some embodiments, do not require or involve surface modification of materials on which cells or patterned.

Still further, in some embodiments, provided technologies do not require and/or do not involve microarray printing (e.g., of cells and/or of surface-modifying agents). In some embodiments, provided technologies do not require and/or do not involve soft-lithography surface patterning technologies such as stamping and/or stenciling. In some embodiments, provided technologies do not require and/or do not involve layer-by-layer assembly of surfaces on which cells are patterned. In some embodiments, provided technologies do not require and/or do not involve microfluidic cell transport. In some embodiments, provided technologies do not require and/or do not involve use of acoustic waves and/or UV or other cross-linking to achieve and/or maintain cell patterning. In some embodiments, provided technologies do not require and/or do not involve polymer blocking of patterned surfaces (e.g., of exposed portions of surfaces on which cells are patterned). In some embodiments, provided technologies do not require and/or do not involve hydrogel encapsulation of patterned cells. Thus, those skilled in the art, reading the present disclosure, will readily appreciate that the present disclosure appreciates and/or identifies the source of problems associated with alternative cell patterning strategies and/or provides simple, efficient, and/or adaptable technologies for achieving cell patterning useful in a variety of contexts. Implementations of apparatus and methods of the present disclosure are useful for a wide range of applications including: tissue design, for example mimicking natural architectures and/or complexity of tissues; monitoring cell-cell interactions, such as creating co-cultures of cells that are known to interact in vivo; investigating cell-microenvironment interactions; and tracking cell migration, proliferation or other changes in response to environmental signals conditions, and/or stimuli.

In some embodiments, the present disclosure describes apparatus. In some embodiments, provided apparatus includes a slab.

In some embodiments, a slab includes an array of wells, which includes at least one well. In some embodiments, an array of wells is defined within a surface of a slab.

In some embodiments, a slab is characterized by an array of wells that form a spatially registered pattern of wells in a surface of a slab.

In some embodiments, a provided apparatus is characterized in that a slab is arranged and constructed to contain wells in a spatial pattern that can be imparted onto cells coated on a substrate by pressing the slab onto the coated cells.

In some embodiments, a provided apparatus is characterized in that a spatial pattern on a slab, includes: a number of wells, a size/dimensions of each well, a total size of a pattern, a total size of a slab, dimensions of a slab, a total number of patterns on a surface of a slab, and spacing of patterns on a surface of a slab.

In some embodiments, each well of an array is characterized by a volume that each well is sized to contain. In some embodiments, a well is sized to contain between about a nanoliter to several hundred microliters. In some embodiments, a well is sized to contain about a nanoliter.

In some embodiments, provided apparatus are characterized in that a slab with wells that are sized to contain a volume in a range of less than about a nL to about 250 μL. In some embodiments, well are sized to contain a volume of less than about a nL, less than about 1.5 nL, less than about 2.0 nL, less than about 2.5 nL, less than about 5.0 nL, less than about 7.5 nL, less than about 10 nL, less than about 15 nL, less than about 20 nL, less than about 25 nL, less than about 50 nL, less than about 75 nL, less than about 100 nL, less than about 150 nL, less than about 200 nL, less than about 250 nL, less than about 500 nL, less than about 750 nL, less than about 1.0 μL, less than about 1.5 μL, less than about 2.0 μL, less than about 2.5 μL, less than about 5.0 μL, less than about 10 μL, less than about 15 μL, less than about 20 μL, less than about 25 μL, less than about 50 μL, less than about 75 μL, less than about 100 μL, less than about 150 μL, less than about 200 μL, or less than about 250 μL.

In some embodiments, an array of wells defined in a surface of a slab are configured to hold a medium containing an environmental signal and/or stimulus in a volume within a well. In some embodiments, an environmental signal and/or stimuli is contained in a medium that is held within at least one well of an array of wells.

In some embodiments, a medium containing an environmental signal and/or stimulus includes an infecting agent and/or a neutralizing agent in a volume within a well.

In some embodiments, an environmental signal and/or stimuli is or comprises an infecting agent. In some embodiments, an environmental signal and/or stimuli that is or comprises an infecting agent is a virus, for example HIV.

In some embodiments, an environmental signal and/or stimuli is or comprises a neutralizing agent. In some embodiments, a neutralizing agent is specific to another environmental signal and/or stimuli. In some embodiments, an environmental signal and/or stimuli that is or comprises a neutralizing agent is an antibody producing cell. In some embodiments, a neutralizing agent produces an antibody. In some embodiments, an antibody produced by a neutralizing agent is a specific response to an infecting agent, for example, an antibody specific to a virus.

In some embodiments, a slab is characterized in that it is manufactured from a moldable material. In some embodiments, a moldable material is or comprises poly(dimethylsiloxane) (PDMS).

In some embodiments, a slab is characterized in that it is manufactured from a deposited material. In some embodiments, a slab is characterized in that it is manufactured from a material that is etched.

In some embodiments, a surface of a slab is substantially planar.

In some embodiments, the present disclosure utilizes a substrate, which may optionally be included in a provided apparatus. In some embodiments, a substrate provides a surface for coating a layer of cells.

In some embodiments, a layer of cells is a monolayer of targets cells. In some embodiments, a substrate surface is uniformly coated with cells. In some embodiments, a substrate uniformly coated with cells is indicative of a uniform cell size distribution. In some embodiments, a uniform cell size distribution is one in which a majority of cells within a population of same cells have a cell diameter within a specified variance. In some embodiments, a majority is more than 50%, 60%, 70%, 75%, 80%, 85%, 90%, 95%, 96%, 97%, 98%, 99%, 99.5%, 99.6%, 99.7%, 99.8%, 99.9% or more cells in a cell population. In some embodiments, a variance in a diameter size is less than about 50%, about 40%, about 30%, about 25%, about 20%, about 15%, about 10%, about 5%, about 4%, about 3%, about 2%, about 1%, about 0.5%, about 0.4%, about 0.3%, about 0.2%, about 0.1% or less. In some embodiments, a substrate uniformly coated with cells is indicative of a substrate surface having about less than 20% surface area not coated with a cell, having about less than 15% surface area not coated with a cell, having about less than 10% surface area not coated with a cell, having about less than 5% surface area not coated with a cell, having about less than 4% surface area not coated with a cell, having about less than 3% surface area not coated with a cell, having about less than 3% surface area not coated with a cell, having about less than 2% surface area not coated with a cell, having about less than 1% surface area not coated with a cell, having about less than 0.5% surface area not coated with a cell or less.

In some embodiments, cells are adhered to a surface of a substrate. In some embodiments, a surface of a substrate is functionalized prior to coating the substrate with a layer of cells. In some embodiments, a substrate is functionalized with cell surface-specific antibodies. In some embodiments, a substrate is functionalized with non-specific adhesion molecules.

In some embodiments, a substrate is made of glass. In some embodiments, a substrate is any standard microscope slide. In some embodiments, a substrate is made of plastic, ceramic, metal, or any material known in the art.

In some embodiments, a surface of a slab is substantially planar.

In some embodiments, provided apparatus includes a slab and a substrate configured to couple with a slab.

In some embodiments, a slab and a substrate couple when a slab and a substrate are reversibly sealed to one another.

In some embodiments, a surface of a substrate coated with a layer of cells is coupled and reversibly sealed to a surface of a slab. In some embodiments, a reversible seal is a substantially fluid tight seal between a surface of a slab and a surface of a substrate.

In some embodiments, a substrate coated with a layer of cells is coupled and reversibly sealed to a slab that is characterized by a spatially registered pattern of wells defined in a surface of a slab.

In some embodiments, a spatially registered pattern of cells is formed on a surface of a substrate when a substrate that is coated with cells is coupled and releasably sealed to a slab that is characterized by a spatially registered pattern of wells defined in a surface of a slab. In some embodiments, a spatially registered pattern of cells is formed when cells are either exposed an array of wells or mechanically disrupted by a surface of a slab defined a spatially registered pattern of wells.

In some embodiments, when a substrate coated with a layer of cells is coupled and reversibly sealed to a slab that is characterized by a spatially registered pattern of wells defined in a surface of a slab an addressable link is formed. In some embodiments, a spatially registered pattern of cells is thereby addressable to a spatially registered pattern of wells and an individual cell is addressable link to a specific well location.

In some embodiments, a substrate coated with cells is coupled and reversibly sealed to a slab that has a spatially registered pattern of wells where at least one well of an array of wells holds a medium containing an environmental signal and/or stimuli. In some embodiments, a spatially registered pattern of cells is formed when cells are mechanically disrupted by a surface of a slab defined a spatially registered pattern of wells or exposed to an array of wells holding a medium containing an environmental signal or stimuli. Accordingly, in some embodiments, when coupled and reversibly sealed, a spatially registered pattern of cells is exposed to an environmental signal and/or stimulus. In some embodiments, provided apparatus and methods are characterized in that when a spatially registered pattern of cells forms where the cells were exposed to an environmental signal and/or stimulus, an addressable link between a spatially registered pattern of wells and a spatially registered pattern of cell is created.

In some embodiments, each individual cell forms an addressable link to a specific well and thereby to a medium containing a specific environmental signal and/or stimuli.

In some embodiments, the present disclosure includes assessing a functional response of an individual cell exposed to environmental signal and/or stimuli.

In some embodiments, the present disclosure includes assessing functional responses of spatially registered pattern of cells that were exposed to environmental signal and/or stimuli. In some embodiments, a correlation between an individual cell and a well location that is addressable can be aggregated to assess a correlation of all cells similarly situated.

In some embodiments, provided methods include providing a substrate coated with a monolayer of cells. In some embodiments, provided methods include providing a slab comprising an array of wells formed into a surface of the slab, wherein the wells a characterized by the presence of an environmental signal and/or stimuli. In some embodiments, each well can contain one or more different environmental signal and/or stimuli of interest, such as an infecting agent and/or a neutralizing agent.

In some embodiments, provided methods include sealing a substrate coated with cells against a surface of a slab. In some embodiments, provided methods include forming a substantially fluid tight seal between a surface of a slab and a surface of a substrate. In some embodiments, provided methods include mechanically disrupting cells according to a pattern formed by wells formed in a surface of a slab, thereby creating a spatially registered pattern of cells. In some embodiments, mechanically disrupting cells occurs where a surface of a slab defining an array of wells contacts a surface of a substrate coated in a monolayer of cells.

In some embodiments, provided methods include incubating cells in wells. In some embodiments, incubating cells in wells when a substrate is releasably sealed to a slab occurs over a period of less than about 12 hours. In some embodiments, an incubation time is about 1 min., about 1.5 min., about 2 min., about 3 min., about 4 min., about 5 min., about 6 min., about 7 min., about 8 min., about 9 min., about 10 min., about 15 min., about 20 min., about 25 min., about 30 min., about 35 min., about 40 min., about 45 min., about 50 min., about 55 min., about 1 hour., about 1.5 hours., about 2 hours, about 3 hours, about 4 hours, about 5 hours, about 6 hours, about 7 hours, about 8 hours, about 9 hours, about 10 hours, about 11 hours, about 12 hours, about 24 hours, about 48 hours, or longer.

In some embodiments, provided methods include releasing a substrate with cells from a slab.

In some embodiments, provided methods include washing away mechanically disrupted cells and leaving cells that are adhered and viable on a coated substrate.

In some embodiments, provided methods include measuring biologically relevant parameters of an individual cell and/or across spatially registered patterns of cells.

In some embodiments, provided methods include linking via an addressable link individual cells to a well of a spatially registered pattern of wells.

In some embodiments, provided methods include assessing change in a response of a cell to an environmental signal or stimuli. In some embodiments, provided methods include assessing an integrated response of a cell to an environmental signal or stimuli.

In some embodiments, provided apparatus and methods are characterized by an ability to study an effect of environmental signal and/or stimuli on cells. In some embodiments, the present apparatus and methods provide a correlation of cells to wells that is useful for assessing functional responses of cells to environmental signal and/or stimuli of interest contained within wells.

In some embodiments, provided apparatus and methods are characterized by an ability to read out an integrated response of cells to an environmental signal and/or stimuli. In some embodiments, provided apparatus and methods measure biologically relevant parameters of cells. In some embodiments, provided apparatus and methods study cell fate and/or phenotype.

In some embodiments, provided apparatus and methods are characterized by an ability to read out a dynamic response of cells to an environmental signal and/or stimuli. In some embodiments, provided apparatus and methods dynamically assess functional responses occurring over a period. In some embodiments, provided apparatus and methods assess responses by incubating cells with environmental signal and/or stimuli for a period. In some embodiments, provided apparatus and methods assess responses through multiple measurements separately acquired over a period. In some embodiments, provided apparatus and methods assess responses of same cells through multiple measurements by iteratively repeating exposure of cell to an environmental signal and/or stimuli over a period. In some embodiments, provided apparatus and methods assess responses of same cells through multiple measurements by repeating exposure with changes to an environmental signal and/or stimuli over a period. As such, provided apparatus and methods are characterized by an ability to provide an understanding of complex behavior of cells in response to environmental signal and/or stimuli.

### BRIEF DESCRIPTION OF THE DRAWING

FIGS. 1(a)-(b) together depict and embodiment of well assisted patterning (WAP). WAP utilizes an array of wells defined in a surface of a slab. Wells in the array are arranged in a spatial pattern. In accordance with the present invention, such a slab can be used as a stamp that, when pressed against cells coated on a substrate surface, mechanically disrupts those cells positioned in points of contact between the slab and the substrate, leaving intact other cells and thereby imparting a spatially registered (with respect to the array of wells) pattern of intact cells on the substrate surface.

FIG. 1(a) depicts an embodiment of a process for creating patterns using a well assisted patterning (WAP) device. As illustrated, a substrate (in this case, a glass slide), is functionalized with an adhesion molecule that retains cells to be patterned on the substrate. In the particular embodiment of FIG. 1(a), a monolayer of cells is formed and substantially uniformly coats part or all of the substrate surface. The cell-coated surface is then reversibly sealed against a surface of a slab containing an array of wells defined therein. Cell patterns are created on the substrate surface by mechanical disruption of those cells positioned at points of contact with the slab. When the substrate is removed from contact with the slab, the pattern is revealed. The substrate can be washed to remove the disrupted cell fragments. As depicted in FIG. 1(a), a pattern of intact cells arranged as islands whose size, shape, and arrangement corresponds to that of the wells in the slab is generated on the substrate surface. Any desired pattern of cells on the substrate can be generated through design of the slabs and its arrangement of wells.

FIG. 1(b) illustrates exemplary cells patterns created through a WAP process as depicted in FIG. 1(a). Specifically, slabs were prepared with different arrangements (e.g., sizes, shapes/orientations) of wells and were used to stamp substrates coated with cells. In particular, slabs with (50 μm wells [left], 100 μm wells [center], and 250 μm wells [right] were utilized. Cells were stained with calcein green and the array was imaged using an epi-fluoresence microscope. The number of cells per pattern was then enumerated. Representative images from three designs are shown, along with histograms of the number of cells per cell island. Scale 50 μm.

FIGS. 2(a)-(b) illustrates control over cell density and pattern coverage. For example, FIG. 2(a) shows that control over the number of cells per pattern was achieved by altering the cell density used to create the monolayer of cells on the surface used to contact the well device. FIG. 2(a) shows a number of cells per pattern as a function of initial cell loading onto substrate to be patterned. A number of cells per pattern can be modulated based on a loading density to achieve a desired final density within the patterns. The number of cells obtained per pattern as a function of initial mono-layer loading density is shown. Representative images are shown below each density, where cells are green and red indicates background antibody stain used to spatially register the array. Scale bar 50 μm.

FIG. 2(b) presents an example image of an entire microscope slide patterned with cells using a slab with wells that are 250 μm in size. As shown, robust coverage of cell patterns was obtained over the entire surface of a microscope slide. Stitched image showing coverage of microscope slide with patterns. Cells were stained with calcein green before imaging. Additionally, the number of cells per pattern could be modulated based on initial loading density of the cells. The above method is spatially robust, allowing for the formation of patterns over an entire microscope slide surface. Using a 250 μm stamp design, for example, 3,328 single wells can be interrogated in parallel, while achieving a larger patterned area in which to interrogate cells.

FIGS. 3(a)-(b) show a measurement of biologically relevant parameters in cell patterns. FIG. 3(a) shows that a migration and proliferation of spatially registered patterns of cells can be determined by imaging patterns over time. As shown, proliferation of spatially registered patterns of cells is calculated based on a total increase in a number of cells per view. Migration of spatially registered patterns of cells is determined by counting cells that migrate out of spatially registered patterned regions. FIG. 3(b) shows images of spatially registered patterns of cells over time shorter time intervals than those shown in FIG. 3(a). In some embodiments, measurements over shorter time intervals allows for a study of dynamic cell behavior, such as quantifying motility of mixed populations of cells stained with different proliferation dyes.

FIGS. 4(a)-(c) illustrates use of provided cell patterning technologies to study cell infection and furthermore to assess neutralization of the infectious agent , e.g., with a spiked antibody. Specifically, FIGS. 4(a)-(b) demonstrates that WAP can quantify infection and neutralization of HIV-pseudovirions using a GHOST cell reporter. Given the critical importance of neutralizing antibodies for the treatment of infectious diseases, and particularly of human immunodeficiency virus (HIV), the results presented in FIGS. 4(a)-(b) confirm the utility and value of technologies provided by the present disclosure. FIG. 4(a) shows that WAP can detect infection of cells with virus in a dose dependent manner. FIG. 4(b) shows that WAP can detect presence of neutralizing antibody (e.g., when included in wells prior to stamping). FIG. 4(c) presents sample images patterned cells exposed to virus in the presence or absence of neutralizing antibody. The total number of infected cells (green) decreases with the addition of neutralizing antibody (b12).

FIGS. 5(a)-(c) illustrates how inventive technology can be used to identify and/or characterize neutralizing antibodies against HIV. As illustrated in FIG. 5(a), antibody secreting cells (such as CHO b12 cells) are deposited in micro-wells of the array, in media that also contains virus. A substrate coated with target cells (e.g., GHOST cells) is then sealed to the array so that the target cells are patterned in a spatial arrangement corresponding to cognate microwells. The substrate and slab are maintained in contact for a period of time. After incubation, the now-patterend GHOST cell slide is removed, and is incubated (e.g., at 37° C. in complete media for 48 hours) to allow reporter signal (e.g., GFP accumulation) to develop. After imaging with a fluorescence microscope, the total number of infected cells per well can be enumerated. Infected cells become green and the total percent of infected cells can be enumerated per pattern (dashed line).

FIG. 5(b) shows the number of antibody secreting cells per micro-well and the resulting percent infected cells per pattern, as enumerated. As can be seen, the infection rate decreases as the number of antibody secreting cells per well increases. In micro-wells containing more antibody secreting cells, the total number of infected GHOST cells per pattern is lower. A natural extension of this assay would be to screen primary B cells from an HIV infected patient in the micro-wells in order to identify cells secreting neutralizing antibodies by detecting patterns of GHOST cells that have a lower infection rate compared to background. In addition to screening primary cells, transformed libraries of antibodies produced in cell lines (e.g. HEK293 or CHO cells) or libraries of yeast variants would also be feasible. A function-based screen for antibodies, combined with single cell retrieval and heavy and light chain antibody sequencing techniques, can rapidly provide the identities of functional members of antibody repertoires.

FIGS. 6(a)-(b) illustrate one embodiment of detection/analysis of latently infected T cells using WAP. That is, as shown, WAP can be applied to study functional characteristics, such as virus production, of cells loaded into micro-wells. ACH2 cells produce HIV virus when stimulated with the cytokine TNFα. As depicted in FIG. 6, ACH2 cells stimulated with TNFα show a greater median percent infection across all loading densities as compared to unstimulated ACH2 cells. Specifically, FIG. 6(a) shows virus producing cells, such as ACH2 cells, are deposited in the micro-well array. These cells produce virus when stimulated with TNFα, which can be contained in the media. The target cell coated slide is incubated with the array, removed, then allowed to incubate while biological signal develops (e.g. GFP accumulation). FIG. 6(a) shows in the bottom panels a representative image of ACH2 cells in the microwell and its corresponding cell pattern, where infected cells are green. FIG. 6(b) shows the median percent infection is plotted as a function of the number of ACH2 cells per micro-well in the presence (+) or absence (−) of TNFα stimulation. The median percent infection is higher in the presence of TNFα for each ACH2 cell count. In addition to assaying viral production capability, functional characteristics of virus producing cells in the micro-wells, such as gene expression, surface marker expression, or secretion of biomolecules of interest could provide functional information about virally infected cells.

FIGS. 7(a)-(b) illustrate a high throughput bi-specific antibody screen performed using cell patterning technology as described herein. Bi-specific antibodies are able to bind two unique antigens, thus, these antibodies have interesting and desirable therapeutic properties. For example, in the context of cancer therapy, bi-specific antibodies may mediate T cell killing of tumor cells by binding a target on the T cell as well as the tumor cell. Feasibility of WAP for high throughput identification and/or characterization of bi-specific antibodies was demonstrated in for antibodies that mediate T cell killing of patterned tumor cells. In this particular assay format, thousands of bi-specific antibody producing cells can be interrogated in parallel. FIG. 7(a) shows cells secreting bi-specific antibody, or media containing bi-specific antibody may be loaded onto the micro-well array. The array is the covered with the cell slide, removed, and incubated with T cells. A functional bi-specific antibody would mediate T cell attachment to tumor cells expressing the correct antigen. After incubation with T cells, tumor cell death could be used to score functional bi-specific antibodies. FIG. 7(b) shows T cells (pink) co-localized with patterned tumor cells expressing the bi-specific receptor, CEA, after incubation with a bi-specific antibody.

FIGS. 8(a)-(b) demonstrate maintenance of a cell pattern generated as described herein. FIG. 8(a) shows a 1% alginate hydrogel was used to encapsulate cell patterns for 48 hours. As shown, cells remain in patterns and do not migrate. FIG. 8(b) shows cells in the patterns continue to proliferate while covered with the hydrogel.

FIGS. 9(a)-(f) show a design of a nano-neutralization assay. A cell-based array infection assay was developed for monitoring thousands of independent HIV neutralization events at the single-cell level in individual nanoliter volume chambers. This assay involved coordination of several biological events; salient technological developments included: a controllable cell pattering method, quantification of antibody secretion kinetics, viral infectivity kinetics, and cell pattern maintenance during infection signal development. FIG. 9(a) shows a schematic of nano-neutralization assay. FIG. 9(b) shows an image of cell patterns created using nano-well assisted cell patterning (top) and histogram of the number of cells per pattern (bottom). FIG. 9(c) shows a quantification of b12 antibody secretion rate from chinese hamster ovary (CHO) cell line (Top). Images show transmitted light (TL), live stain (Live), and corresponding protein array signal (IgG) for a representative well (Bottom). To verify titers of antibody relevant to neutralization could be achieved using antibody-secreting cells in the wells, the number of antibody secreting cells needed to produce the required amount of antibody given the well geometry to allow for virus neutralization in each well was determined. A Chinese hamster ovary (CHO) cell line producing the well-characterized neutralizing antibody b12 was stained with calcein green and loaded onto the PDMS device, and microengraving was used to quantify antibody secretion to determine secretion rate as previously described (see Han, K. et al., “Parallel Measurement of Dynamic Changes in Translation Rates in Single Cells,” 11 Nat Meth, 86-93 (2013), which is hereby incorporated by reference in its entirety herein. FIG. 9(d) shows a serial dilution curve of pseudovirus. The number of infected GHOST cells per pattern was determined as a function of virus dilution loaded onto the nanowells. Relative infectivity is plotted as a function of virus dilution. The median and SEM are plotted. Error bars, s.d. FIG. 9(e) shows kinetics of a virus infection were determined by infecting GHOST cells for the indicated amount of time in the nanowells. Relative infectivity is plotted as a function of time. Using the method of Platt et al. 2010, the t_{1/2} of infection was determined to be 123 minutes. The mean and SEM are plotted. Error bars, s.d. FIG. 9(f) shows an addition of focal adhesion kinase inhibitor to media prevents loss of cell patterns. Representative images show a block of patterns at time 0 hr and 24 hrs post patterning. Cells are stained with calcein violet (yellow) and background pattern is shown for each well (purple). Scale bar 100 μm.

FIGS. 10(a)-(b) show an implementation of nano-neutralization assay using cell lines producing the neutralizing antibodies b12, b6, 2F5, 2G12 or the non-neutralizing antibody 4D20. Nano-neutralization assay allows for specific and sensitive detection of neutralizing antibody producing cell lines. FIG. 10(a) shows sample images of nano-well array loaded with stained CHO b12 (cyan) or CHO b6 (pink) cells. For cell pattern images of target GHOST cells, live stain is red, infected cells are green, and the background of each pattern is blue. To screen for neutralization of virus by neutralizing antibody secreting cells, we loaded the nanowells with cell lines secreting either the neutralizing antibody b12 or the non-neutralizing antibody b6, and imaged the array using an epifluoresence microscope in order to determine well occupancy. After the incubation, cell arrays were removed, washed, and placed in media containing FAK inhibitor for 48 hours in order to maintain spatial registration of cell patterns. FIG. 10(b) shows quantification of relative infectivity as a function of number of CHO b6 (red bars) or CHO b12 (black bars) per nano-well. The number of measured events is indicated on each bar, error bars were propagated from the SEM and indicated comparisons between black bars. ***, p<0.001, *, p<0.05, Kruskal-Wallis test with Dunn's multiple comparison post-test. The relative infectivity decreased significantly as a function of the number of b12 secreting cells, but not b6 cells, per nanowell. Additionally, as low as one to two b12 secreting cells per well led to a significant decrease in the infection rate of cells (p=0.0307).

FIGS. 11(a)-(b) show an ROC curve analysis of nano-neutralization assay. In order to assess the performance of this screen for the classification of antibody secreting cells into those that produce neutralizing antibodies (neutralizers) versus those that do not produce neutralizing antibodies (non-neutralizers), a receiver operating characteristic (ROC) was generated. (See Baker, S. G., “The Central Role of Receiver Operating Characteristic (ROC) Curves in Evaluating Tests for the Early Detection of Cancer,” 95 J. National Cancer Institute, 511-515 (2003), which is incorporated by reference in its entirety herein). FIG. 11(a) shows ROC curves for each antibody secreting cell line screened using the nano-neutralization assay. To determine if a decrease in infection against a background population of non-specific antibody secreting cells could be detected, as well as determine if we could detect neutralization against another viral epitope, nano-neutralization assay were performed with additional antibody-secreting cell lines producing the non-neutralizing antibody 4D20 and the gp41 directed neutralizing antibody 2F5. ROC curves for all antibody secreting cell lines tested indicated that the screen allows for the identification of neutralizing, non-neutralizing and weakly neutralizing antibodies, where the true positive rates are higher for antibody cell lines producing higher affinity neutralizing antibodies (i.e.: b12). FIG. 11(b) shows a calculation of positive predictive value of b12 screen for indicated infectivity thresholds and neutralizer prevalence. Infectivity thresholds were determined from wells containing no antibody secreting cells. The positive predictive value (PPV) and negative predictive value (NPV) for several thresholds of infection rate given a variable prevalence of neutralizers in a given population of antibody secreting cells were calculated. The goal of the screen is to enrich in neutralizers while minimizing false positives, thus, we would aim to increase the PPV rate while also maintaining a high NPV. It was found that the PPV was highest (27%) for a population prevalence of 10% neutralizers when the threshold was the most stringent. The above result would suggest that this screen would be very well suited for screening libraries of antibody secreting cells, where oversampling the library by at least 4-fold would result in identification of all neutralizers present. Alternatively, increasing the prevalence of neutralizers to 50% of the population screened would lead to dramatic increases in PPV rate, which indicates that this screen could also be implemented on pre-enriched populations of antibody secreting cells. Finally, the addition of TAK779 to the virus prior to incubating with cells drastically improves the sensitivity and specificity of the assay across all thresholds. Data obtained from b12 secreting cells was used to determine the optimal cell count and the optimal infectivity threshold for classifying neutralizers and non-neutralizers.

FIGS. 12(a)-(b) show infectivity of pseudovirus assessed by micro-titre plate infection and on glass. FIG. 12(a) shows virus infectivity assessed by flow cytometry. The characterization of the infectivity of HIV pseudovirus in nanowells was required to determine a comparison to conventional neutralization assays. GHOST cells were used as a standard cell-line used for microtitre-plate based neutralization assays (see Morner, A. et al., “Primary Human Immunodeficiency Virus Type 2 (HIV-2) Isolates, Like HIV-1 Isolates, Frequently Use CCR5 but Show Promiscuity in Coreceptor Usage,” J. Virology, 2343-2349 (1999), which is hereby incorporated by reference in its entirety herein), as cells. This cell line produces green fluorescent protein (gfp) when infected with HIV, thus providing a fluorescent signal for the quantification of infection. Pseudovirus was produced according to a previously described protocol (Greene et al.), and virions were harvested, spin concentrated ten-fold, and their infectivity characterized by flow cytometry. Representative flow plots showing uninfected GHOST cells and GHOST cells infected with psuedovirus (10× dilution). The table displays infectivity for virus harvested on day 1 and day 2 after transfection and used at the indicated dilution. Background infectivity was calculated as 0.4% for GHOST cells exposed to no virus.

FIG. 12(b) shows virus infectivity on glass. Left: Representative image of infected cells (green) on glass, where red dashed lines show locations of wells. Right: Histogram of percent infectivity for wells analyzed. Virions were found more infective in day 2 supernatants, with an average infectivity rate of 56.25% at a dilution of 1:10. For all virus experiments using nanowells, virus preparations from the same day and batch were used for a given experiment. To determine if infection of cells could be detected using nanowell assisted cell patterning, nanowell arrays loaded with virus before sealing the array and allowed infection to proceed for 3 hours at 37° C. Infection of patterned cells were measured at a median rate of 30% and the relative infection of patterned cells decreased with virus dilution as shown in FIG. 9(d).

FIG. 13 shows that percent infection per pattern does not vary with the cell density. The median percent infection for a range of cell densities is shown. Error bars, S.E.M. The median infection rate did not vary significantly as a function of GHOST cell density. To characterize the kinetics of viral infection in the nanowells, virus was loaded onto the nanowell array and arrays were sealed for 60, 120, 180, or 240 minutes, as shown in FIG. 9(e). Kinetic data for infection were fit as previously described (see Platt, E. J. et al., “Kinetic Factors Control Efficiencies of Cell Entry, Efficacies of Entry Inhibitors, and Mechanisms of Adaptation of Human Immunodeficiency Virus,” 79 J. Virology, 4347-4356 (2005), which is incorporated by reference in its entirety herein), and we found the t_{1/2} of infection to be 126 minutes, which is on par with what has been reported previously (see Reeves, J. D., “Sensitivity of HIV-1 to Entry Inhibitors Correlates with Envelope/Coreceptor Affinity, Receptor Density, and Fusion Kinetics,” 99 PNAS, 16249-16254 (2002), which is hereby incorporated by reference in its entirety herein). Taken together, these data indicated that virus was capable of infecting cells, signal readout was proportional to virus dilution tested, and infection rate was comparable to those previously reported in the literature.

FIG. 14 shows an effect of ROCK inhibitor and FAK inhibitor on pseudovirus infection of cultured GHOST cells. FIG. 14(a) shows a percent infection as a function of FAK inhibitor (PF-573228) concentration (μM). FIG. 14(b) shows percent infection as a function of ROCK inhibitor (Y-27632) concentration (μM). Error bars, S.E.M.

One significant advantage afforded by WAP is the ability to an addressable link with stimuli with delayed signal development in cells, such as infection, proliferation, or migration. For implementation of the nano-neutralization assay, a technique for preventing pattern disruption was developed over time due to proliferation and migration of GHOST cells over the course of infection signal development. The effect of several media additives and hydrogels for the maintenance of patterns was tested. It was found that the addition of hydrogels, such as alginate and pluronic acid, as cell encapsulation materials did not allow for pattern maintenance while allowing for infection signal development (data not shown). The addition of a focal adhesion kinase inhibitor (PF573228) or Rho-kinase inhibitor (Y-27632), both shown to inhibit cell motility but not adhesion (see Loerke, D. et al., “Quantitative Imaging of Epithelial Cell Scattering Identifies Specific Inhibitors of Cell Motility and Cell-Cell Dissociation,” 5 Science Signaling, rs5-rs5 (2012), which is hereby incorporated by reference in its entirety, to cell culture media at concentrations would not significantly affect infection would allow for pattern maintenance.

FIGS. 15(a)-(c) show that addition of a focal adhesion kinase (FAK) inhibitor (PF-573228) allowed for pattern maintenance over time. The addition of 10 μM FAK inhibitor resulted in individual pattern maintenance for the time required for signal development by preventing both the migration and proliferation of attached cells. FIG. 15(a) shows representative images of cell patterns in the presence or absence of FAK inhibitor after 24 or 48 hours of growth. Cells were stained with calcein green before imaging. Scale bar 100 μm. FIG. 15(b) shows an addition of FAK inhibitor reduced proliferation of patterned cells, where proliferation was calculated as the ratio of cells per block (Nt) divided by the initial cells per block (Nto). FIG. 15(c) shows an addition of FAK inhibitor reduced migration of patterned cells. Migration was defined as the number of cells migrating out of patterns. **** p<0.0001, Mann-Whitney U test. These data show that the addition of FAK inhibitor allows for the tight coupling of nanowell and cognate cell pattern, which drastically extends the options for signal readout in this cell-based assay.

FIG. 16 shows a histogram of area under the curve (AUC) for nano-neutralization assays performed with antibody secreting cell lines. The AUC is plotted, along with the calculated standard error. The area under a straight line, 0.5, is shown as the dashed line. Based on calculated area under the curve (AUC) values for each cell line, we can use the nano-neutralization assay to identify b12 neutralizers with 70.95% confidence and 2F5 with 66.06%, while 4D20 cells, the non-neutralizing antibody producing cell line, is close to 50%. Interestingly, we found that the non-neutralizer b6 could also be classified with 63.67% confidence. The ability of b6 to bind with weak affinity to gp120 (see Pantophlet, R. et al., “Fine Mapping of the Interaction of Neutralizing and Nonneutralizing Monoclonal Antibodies with the CD4 Binding Site of Human Immunodeficiency Virus Type 1 Gp120,” 77 J. Virology, 642-658 (2003), which is incorporated by reference herein in its entirety, could have contributed to the increase above background as compared to 4D20. Taken together with high PPR rates for b12, these data show that the nano-neutralization screen can be implemented to identify neutralizing antibody secreting cells with a high specificity, with a bias towards identification of strongly neutralizing antibodies.

FIG. 17 shows a manual count of infected cells per well (top), a manual count of CHO-b12 cells loaded in 250 μm wells (center), and a manual count of CHO-b12 cells loaded in 100 μm wells (bottom).

FIG. 18 shows an estimation of antibody concentration using a calculated secretion rate for 250 μm nanowell and an assumed constant secretion rate of 50 molecules/cell*second and one CHO cell per well. As shown a concentration of antibody in each nanowell as a function of time was determined. A well containing one cell was found to be able to reach a concentration of 0.014 ug/mL in a time of three hours, which is in the low ug/mL range of reported IC₅₀ values for b12. (See Moldt, B. et al., “Highly Potent HIV-Specific Antibody Neutralization in Vitro Translates Into Effective Protection Against Mucosal SHIV Challenge in Vivo,” 109 Proceedings of the National Academy of Sciences, 18921-18925 (2012); Blish, C. A. et al., “Enhancing Exposure of HIV-1 Neutralization Epitopes Through Mutations in Gp41,” PLoS Medicine, 1-14 (2008); and Burton, D. R. et al., “Efficient Neutralization of Primary Isolates of HIV-1 by a Recombinant Human Monoclonal Antibody,” Science, 1-4 (1994), each of which is incorporated herein by reference it their entirety.

FIG. 19 shows a percent infection as a function of TAK779 concentration (μM). A nanowell device was loaded with pseudovirus and sealed with cells for 3 hours. To slow viral entry process to ensure high titers of antibody were reached prior to infection, sub-nanomolar concentration of TAK779 was added with the virus, as previous studies have demonstrated that TAK779 lowers the effective CCR5 concentration and thus, slows HIV-1 entry. (See Baba, M., “A Small-Molecule, Nonpetide CCR5 Antagonist with Highly Potent and Selective Anti-HIV-1 Activity,” PNAS, 1-6 (1999) and Platt, E. J. et al., “Rapid Dissociation of HIV-1 From Cultured Cells Severely Limits Infectivity Assays, Causes the Inactivation Ascribed to Entry Inhibitors, and Masks the Inherently High Level of Infectivity of Virions,” 84 J. Virology, 3106-3110 (2010), both of which are incorporated by reference in their entirety herein.

FIG. 20 shows area under the curve (AUC) values for varying criteria of assigning control and experimental wells for b12 antibody secreting cells. It was determined that higher true positive rates could be obtained when neutralizers were determined from nanowells containing >5 cells, which could indicate that higher antibody secretion rates would improve screening sensitivity.

### DEFINITIONS

In some embodiments, provided apparatus and/or methods are characterized in that they allow study of cell behavior in a variety of simulated biological environments and/or permit high-throughput analysis of cell attributes and/or responses, and/or those of agents that affect them. In order for the present disclosure to be more readily understood, certain terms are first defined below. Additional definitions for the following terms and other terms are set forth throughout the specification.

In this application, unless otherwise clear from context, the term “a” may be understood to mean “at least one.” As used in this application, the term “or” may be understood to mean “and/or.” In this application, the terms “comprising” and “including” may be understood to encompass itemized components or steps whether presented by themselves or together with one or more additional components or steps. Unless otherwise stated, the terms “about” and “approximately” may be understood to permit standard variation as would be understood by those of ordinary skill in the art. Where ranges are provided herein, the endpoints are included. As used in this application, the term “comprise” and variations of the term, such as “comprising” and “comprises,” are not intended to exclude other additives, components, integers or steps.

As used in this application, the terms “about” and “approximately” are used as equivalents. Any numerals used in this application with or without about/approximately are meant to cover any normal fluctuations appreciated by one of ordinary skill in the relevant art. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

“Administration”: As used herein, the term “administration” refers to the administration of a composition to a subject or system. Administration to an animal subject (e.g., to a human) may be by any appropriate route. For example, in some embodiments, administration may be bronchial (including by bronchial instillation), buccal, enteral, interdermal, intra-arterial, intradermal, intragastric, intramedullary, intramuscular, intranasal, intraperitoneal, intrathecal, intravenous, intraventricular, mucosal, nasal, oral, rectal, subcutaneous, sublingual, topical, tracheal (including by intratracheal instillation), transdermal, vaginal and vitreal. In some embodiments, administration may involve intermittent dosing. In some embodiments, administration may involve continuous dosing (e.g., perfusion) for at least a selected period of time.

“Affinity”: As is known in the art, “affinity” is a measure of the tightness with a particular ligand binds to its partner. Affinities can be measured in different ways. In some embodiments, affinity is measured by a quantitative assay. In some such embodiments, binding partner concentration may be fixed to be in excess of ligand concentration so as to mimic physiological conditions. Alternatively or additionally, in some embodiments, binding partner concentration and/or ligand concentration may be varied. In some such embodiments, affinity may be compared to a reference under comparable conditions (e.g., concentrations).

“Agent”: As used herein, the term “agent” may refer to a compound or entity of any chemical class including, for example, polypeptides, nucleic acids, saccharides, lipids, small molecules, metals, or combinations thereof. As will be clear from context, in some embodiments, an agent can be or comprise a cell or organism, or a fraction, extract, or component thereof. In some embodiments, an agent is agent is or comprises a natural product in that it is found in and/or is obtained from nature. In some embodiments, an agent is or comprises one or more entities that is man-made in that it is designed, engineered, and/or produced through action of the hand of man and/or is not found in nature. In some embodiments, an agent may be utilized in isolated or pure form; in some embodiments, an agent may be utilized in crude form. In some embodiments, potential agents are provided as collections or libraries, for example that may be screened to identify or characterize active agents within them. Some particular embodiments of agents that may be utilized in accordance with the present invention include small molecules, antibodies, antibody fragments, aptamers, siRNAs, shRNAs, DNA/RNA hybrids, antisense oligonucleotides, ribozymes, peptides, peptide mimetics, small molecules, etc. In some embodiments, an agent is or comprises a polymer. In some embodiments, an agent is not a polymer and/or is substantially free of any polymer. In some embodiments, an agent contains at least one polymeric moiety. In some embodiments, an agent lacks or is substantially free of any polymeric moiety.

“Analog”: As used herein, the term “analog” refers to a substance that shares one or more particular structural features, elements, components, or moieties with a reference substance. Typically, an “analog” shows significant structural similarity with the reference substance, for example sharing a core or consensus structure, but also differs in certain discrete ways. In some embodiments, an analog is a substance that can be generated from the reference substance by chemical manipulation of the reference substance. In some embodiments, an analog is a substance that can be generated through performance of a synthetic process substantially similar to (e.g., sharing a plurality of steps with) one that generates the reference substance. In some embodiments, an analog is or can be generated through performance of a synthetic process different from that used to generate the reference substance

“Antibody”: As used herein, the term “antibody” refers to a polypeptide that includes canonical immunoglobulin sequence elements sufficient to confer specific binding to a particular target antigen. As is known in the art, intact antibodies as produced in nature are approximately 150 kD tetrameric agents comprised of two identical heavy chain polypeptides (about 50 kD each) and two identical light chain polypeptides (about 25 kD each) that associate with each other into what is commonly referred to as a “Y-shaped” structure. Each heavy chain is comprised of at least four domains (each about 110 amino acids long)—an amino-terminal variable (VH) domain (located at the tips of the Y structure), followed by three constant domains: CH1, CH2, and the carboxy-terminal CH3 (located at the base of the Y's stem). A short region, known as the “switch”, connects the heavy chain variable and constant regions. The “hinge” connects CH2 and CH3 domains to the rest of the antibody. Two disulfide bonds in this hinge region connect the two heavy chain polypeptides to one another in an intact antibody. Each light chain is comprised of two domains—an amino-terminal variable (VL) domain, followed by a carboxy-terminal constant (CL) domain, separated from one another by another “switch”. Intact antibody tetramers are comprised of two heavy chain-light chain dimers in which the heavy and light chains are linked to one another by a single disulfide bond; two other disulfide bonds connect the heavy chain hinge regions to one another, so that the dimers are connected to one another and the tetramer is formed. Naturally-produced antibodies are also glycosylated, typically on the CH2 domain. Each domain in a natural antibody has a structure characterized by an “immunoglobulin fold” formed from two beta sheets (e.g., 3-, 4-, or 5-stranded sheets) packed against each other in a compressed antiparallel beta barrel. Each variable domain contains three hypervariable loops known as “complement determining regions” (CDR1, CDR2, and CDR3) and four somewhat invariant “framework” regions (FR1, FR2, FR3, and FR4). When natural antibodies fold, the FR regions form the beta sheets that provide the structural framework for the domains, and the CDR loop regions from both the heavy and light chains are brought together in three-dimensional space so that they create a single hypervariable antigen binding site located at the tip of the Y structure. Amino acid sequence comparisons among antibody polypeptide chains have defined two light chain (κ and λ) classes, several heavy chain (e.g., μ, γ, α, ε, δ) classes, and certain heavy chain subclasses (α1, α2, γ1, γ2, γ3, and γ4). Antibody classes (IgA [including IgA1, IgA2], IgD, IgE, IgG [including IgG1, IgG2, IgG3, IgG4], IgM) are defined based on the class of the utilized heavy chain sequences. For purposes of the present invention, in certain embodiments, any polypeptide or complex of polypeptides that includes sufficient immunoglobulin domain sequences as found in natural antibodies can be referred to and/or used as an “antibody”, whether such polypeptide is naturally produced (e.g., generated by an organism reacting to an antigen), or produced by recombinant engineering, chemical synthesis, or other artificial system or methodology. In some embodiments, an antibody is monoclonal; in some embodiments, an antibody is monoclonal. In some embodiments, an antibody has constant region sequences that are characteristic of mouse, rabbit, primate, or human antibodies. In some embodiments, an antibody sequence elements are humanized, primatized, chimeric, etc, as is known in the art. Moreover, the term “antibody” as used herein, will be understood to encompass (unless otherwise stated or clear from context) can refer in appropriate embodiments to any of the art-known or developed constructs or formats for capturing antibody structural and functional features in alternative presentation. For example, in some embodiments, the term can refer to bi- or other multi-specific (e.g., zybodies, etc) antibodies, Small Modular ImmunoPharmaceuticals (“SMIPs™”), single chain antibodies, cameloid antibodies, and/or antibody fragments. In some embodiments, an antibody may lack a covalent modification (e.g., attachment of a glycan) that it would have if produced naturally. In some embodiments, an antibody may contain a covalent modification (e.g., attachment of a glycan, a payload [e.g., a detectable moiety, a therapeutic moiety, a catalytic moiety, etc], or other pendant group [e.g., poly-ethylene glycol, etc].

“Antigen”: The term “antigen”, as used herein, refers to an agent that elicits an immune response; and/or (ii) an agent that binds to a T cell receptor (e.g., when presented by an MHC molecule) or to an antibody. In some embodiments, an antigen elicits a humoral response (e.g., including production of antigen-specific antibodies); in some embodiments, an elicits a cellular response (e.g., involving T-cells whose receptors specifically interact with the antigen). In some embodiments, and antigen binds to an antibody and may or may not induce a particular physiological response in an organism. In general, an antigen may be or include any chemical entity such as, for example, a small molecule, a nucleic acid, a polypeptide, a carbohydrate, a lipid, a polymer (in some embodiments other than a biologic polmer [e.g., other than a nucleic acid or amino acid polymer) etc. In some embodiments, an antigen is or comprises a polypeptide. In some embodiments, an antigen is or comprises a glycan. Those of ordinary skill in the art will appreciate that, in general, an antigen may be provided in isolated or pure form, or alternatively may be provided in crude form (e.g., together with other materials, for example in an extract such as a cellular extract or other relatively crude preparation of an antigen-containing source). In some embodiments, antigens utilized in accordance with the present invention are provided in a crude form. In some embodiments, an antigen is a recombinant antigen.

“Associated with”: Two events or entities are “associated” with one another, as that term is used herein, if the presence, level and/or form of one is correlated with that of the other. For example, a particular entity (e.g., polypeptide) is considered to be associated with a particular disease, disorder, or condition, if its presence, level and/or form correlates with incidence of and/or susceptibility to the disease, disorder, or condition (e.g., across a relevant population). In some embodiments, two or more entities are physically “associated” with one another if they interact, directly or indirectly, so that they are and remain in physical proximity with one another. In some embodiments, two or more entities that are physically associated with one another are covalently linked to one another; in some embodiments, two or more entities that are physically associated with one another are not covalently linked to one another but are non-covalently associated, for example by means of hydrogen bonds, van der Waals interaction, hydrophobic interactions, magnetism, and combinations thereof.

“Binding”: It will be understood that the term “binding”, as used herein, typically refers to a non-covalent association between or among two or more entities. “Direct” binding involves physical contact between entities or moieties; indirect binding involves physical interaction by way of physical contact with one or more intermediate entities. Binding between two or more entities can typically be assessed in any of a variety of contexts—including where interacting entities or moieties are studied in isolation or in the context of more complex systems (e.g., while covalently or otherwise associated with a carrier entity and/or in a biological system or cell).

“Binding agent”: In general, the term “binding agent” is used herein to refer to any entity that binds to a target of interest as described herein. In many embodiments, a binding agent of interest is one that binds specifically with its target in that it discriminates its target from other potential binding partners in a particular interaction contect. In general, a binding agent may be or comprise an entity of any chemical class (e.g., polymer, non-polymer, small molecule, polypeptide, carbohydrate, lipid, nucleic acid, etc). In some embodiments, a binding agent is a single chemical entity. In some embodiments, a binding agent is a complex of two or more discrete chemical entities associated with one another under relevant conditions by non-covalent interactions. For example, those skilled in the art will appreciate that in some embodiments, a binding agent may comprise a “generic” binding moiety (e.g., one of biotin/avidin/streptaviding and/or a class-specific antibody) and a “specific” binding moiety (e.g., an antibody or aptamers with a particular molecular target) that is linked to the partner of the generic biding moiety. In some embodiments, such an approach can permit modular assembly of multiple binding agents through linkage of different specific binding moieties with the same generic binding poiety partner. In some embodiments, binding agents are or comprise polypeptides (including, e.g., antibodies or antibody fragments). In some embodiments, binding agents are or comprise small molecules. In some embodiments, binding agents are or comprise nucleic acids. In some embodiments, binding agents are aptamers. In some embodiments, binding agents are polymers; in some embodiments, binding agents are not polymers. In some embodiments, binding agents are non-polymeric in that they lack polymeric moieties. In some embodiments, binding agents are or comprise carbohydrates. In some embodiments, binding agents are or comprise lectins. In some embodiments, binding agents are or comprise peptidomimetics. In some embodiments, binding agents are or comprise scaffold proteins. In some embodiments, binding agents are or comprise mimeotopes. In some embodiments, binding agents are or comprise stapled peptides. In certain embodiments, binding agents are or comprise nucleic acids, such as DNA or RNA.

“Biocompatible”: The term “biocompatible”, as used herein, refers to materials that do not cause significant harm to living tissue when placed in contact with such tissue, e.g., in vivo. In certain embodiments, materials are “biocompatible” if they are not toxic to cells. In certain embodiments, materials are “biocompatible” if their addition to cells in vitro results in less than or equal to 20% cell death, and/or their administration in vivo does not induce significant inflammation or other such adverse effects.

“Biologically active”: As used herein, the phrase “biologically active” refers to a substance that has activity in a biological system (e.g., in a cell (e.g., isolated, in culture, in a tissue, in an organism), in a cell culture, in a tissue, in an organism, etc.). For instance, a substance that, when administered to an organism, has a biological effect on that organism, is considered to be biologically active. It will be appreciated by those skilled in the art that often only a portion or fragment of a biologically active substance is required (e.g., is necessary and sufficient) for the activity to be present; in such circumstances, that portion or fragment is considered to be a “biologically active” portion or fragment.

“Characteristic portion”: As used herein, the term “characteristic portion” is used, in the broadest sense, to refer to a portion of a substance whose presence (or absence) correlates with presence (or absence) of a particular feature, attribute, or activity of the substance. In some embodiments, a characteristic portion of a substance is a portion that is found in the substance and in related substances that share the particular feature, attribute or activity, but not in those that do not share the particular feature, attribute or activity. In certain embodiments, a characteristic portion shares at least one functional characteristic with the intact substance. For example, in some embodiments, a “characteristic portion” of a protein or polypeptide is one that contains a continuous stretch of amino acids, or a collection of continuous stretches of amino acids, that together are characteristic of a protein or polypeptide. In some embodiments, each such continuous stretch generally contains at least 2, 5, 10, 15, 20, 50, or more amino acids. In general, a characteristic portion of a substance (e.g., of a protein, antibody, etc.) is one that, in addition to the sequence and/or structural identity specified above, shares at least one functional characteristic with the relevant intact substance. In some embodiments, a characteristic portion may be biologically active.

“Comparable”: The term “comparable”, as used herein, refers to two or more agents, entities, situations, sets of conditions, etc that may not be identical to one another but that are sufficiently similar to permit comparison therebetween so that conclusions may reasonably be drawn based on differences or similarities observed. In some embodiments, comparable sets of conditions, circumstances, individuals, or populations are characterized by a plurality of substantially identical features and one or a small number of varied features. Those of ordinary skill in the art will understand, in context, what degree of identity is required in any given circumstance for two or more such agents, entities, situations, sets of conditions, etc to be considered comparable. For example, those of ordinary skill in the art will appreciate that sets of circumstances, individuals, or populations are comparable to one another when characterized by a sufficient number and type of substantially identical features to warrant a reasonable conclusion that differences in results obtained or phenomena observed under or with different sets of circumstances, individuals, or populations are caused by or indicative of the variation in those features that are varied.

“Corresponding to”: As used herein, the term “corresponding to” is often used to designate a structural element or moiety in an agent of interest that shares a position (e.g., in three-dimensional space or relative to another element or moiety) with one present in an appropriate reference agent. For example, in some embodiments, the term is used to refer to position/identity of a residue in a polymer, such as an amino acid residue in a polypeptide or a nucleotide residue in a nucleic acid. Those of ordinary skill will appreciate that, for purposes of simplicity, residues in such a polymer are often designated using a canonical numbering system based on a reference related polymer, so that a residue in a first polymer “corresponding to” a residue at position 190 in the reference polymer, for example, need not actually be the 190^{th} residue in the first polymer but rather corresponds to the residue found at the 190^{th} position in the reference polymer; those of ordinary skill in the art readily appreciate how to identify “corresponding” amino acids, including through use of one or more commercially-available algorithms specifically designed for polymer sequence comparisons.

“Detection entity”: The term “detection entity” as used herein refers to any element, molecule, functional group, compound, fragment or moiety that is detectable. In some embodiments, a detection entity is provided or utilized alone. In some embodiments, a detection entity is provided and/or utilized in association with (e.g., joined to) another agent. Examples of detection entities include, but are not limited to: various ligands, radionuclides (e.g., ³H, ¹⁴C, ¹⁸F, ¹⁹F, ³²P, ³⁵S, ¹³⁵I, ¹²⁵I, ¹²³I, ⁶⁴Cu, ¹⁸⁷Re, ¹¹¹In, ⁹⁰Y, ^{99m}Tc, ¹⁷⁷Lu, ⁸⁹Zr etc.), fluorescent dyes (for specific exemplary fluorescent dyes, see below), chemiluminescent agents (such as, for example, acridinum esters, stabilized dioxetanes, and the like), bioluminescent agents, spectrally resolvable inorganic fluorescent semiconductors nanocrystals (i.e., quantum dots), metal nanoparticles (e.g., gold, silver, copper, platinum, etc.) nanoclusters, paramagnetic metal ions, enzymes (for specific examples of enzymes, see below), colorimetric labels (such as, for example, dyes, colloidal gold, and the like), biotin, dioxigenin, haptens, and proteins for which antisera or monoclonal antibodies are available.

“Determine”: Many methodologies described herein include a step of “determining”. Those of ordinary skill in the art, reading the present specification, will appreciate that such “determining” can utilize or be accomplished through use of any of a variety of techniques available to those skilled in the art, including for example specific techniques explicitly referred to herein. In some embodiments, determining involves manipulation of a physical sample. In some embodiments, determining involves consideration and/or manipulation of data or information, for example utilizing a computer or other processing unit adapted to perform a relevant analysis. In some embodiments, determining involves receiving relevant information and/or materials from a source. In some embodiments, determining involves comparing one or more features of a sample or entity to a comparable reference.

“Infecting agent”: As used herein, the phrase “infecting agent” refers to an agent or organism that invades a host organism, tissue, or cell. In many embodiments, an infecting agent is characterized in that it multiplies (or is multiplied) in the host, organism, tissue, or cell. In some embodiments, infection with an infecting agent results in one or more symptoms. In some embodiments, one or more symptoms may result from activity of an agent (e.g., a toxin) produced by the infecting agent. In some embodiments, one or more symptoms may result from replication of the infecting agent. In some embodiments, one or more symptoms may reflect reaction of host organism(s), tissue(s), and/or cell(s) to the infecting agent and/or one or more products it generates.

“Marker”: A marker, as used herein, refers to an entity or moiety whose presence or level is a characteristic of a particular state or event. In some embodiments, presence or level of a particular marker may be characteristic of presence or stage of a disease, disorder, or condition. To give but one example, in some embodiments, the term refers to a gene expression product that is characteristic of a particular tumor, tumor subclass, stage of tumor, etc. Alternatively or additionally, in some embodiments, a presence or level of a particular marker correlates with activity (or activity level) of a particular signaling pathway, for example that may be characteristic of a particular class of tumors. The statistical significance of the presence or absence of a marker may vary depending upon the particular marker. In some embodiments, detection of a marker is highly specific in that it reflects a high probability that the tumor is of a particular subclass. Such specificity may come at the cost of sensitivity (i.e., a negative result may occur even if the tumor is a tumor that would be expected to express the marker). Conversely, markers with a high degree of sensitivity may be less specific that those with lower sensitivity. According to the present invention a useful marker need not distinguish tumors of a particular subclass with 100% accuracy.

“Modulator”: The term “modulator” is used to refer to an entity whose presence or level in a system in which an activity of interest is observed correlates with a change in level and/or nature of that activity as compared with that observed under otherwise comparable conditions when the modulator is absent. In some embodiments, a modulator is an activator, in that activity is increased in its presence as compared with that observed under otherwise comparable conditions when the modulator is absent. In some embodiments, a modulator is an antagonist or inhibitor, in that activity is reduced in its presence as compared with otherwise comparable conditions when the modulator is absent. In some embodiments, a modulator interacts directly with a target entity whose activity is of interest. In some embodiments, a modulator interacts indirectly (i.e., directly with an intermediate agent that interacts with the target entity) with a target entity whose activity is of interest. In some embodiments, a modulator affects level of a target entity of interest; alternatively or additionally, in some embodiments, a modulator affects activity of a target entity of interest without affecting level of the target entity. In some embodiments, a modulator affects both level and activity of a target entity of interest, so that an observed difference in activity is not entirely explained by or commensurate with an observed difference in level.

“Neutralizing agent”: As used herein, the phrase “neutralizing agent” refers to an agent that reduces (e.g., in incidence and/or severity) and/or delays one or more features, characteristics, or symptoms of a damaging agent (e.g., an infecting agent). In some embodiments, a neutralizing agent is or comprises a therapeutic agent.

“Pharmaceutical composition”: As used herein, the term “pharmaceutical composition” refers to an active agent, formulated together with one or more pharmaceutically acceptable carriers. In some embodiments, active agent is present in unit dose amount appropriate for administration in a therapeutic regimen that shows a statistically significant probability of achieving a predetermined therapeutic effect when administered to a relevant population. In some embodiments, pharmaceutical compositions may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; transdermally; or nasally, pulmonary, and to other mucosal surfaces.

“Reference”: The term “reference” is often used herein to describe a standard or control agent, individual, population, sample, sequence or value against which an agent, individual, population, sample, sequence or value of interest is compared. In some embodiments, a reference agent, individual, population, sample, sequence or value is tested and/or determined substantially simultaneously with the testing or determination of the agent, individual, population, sample, sequence or value of interest. In some embodiments, a reference agent, individual, population, sample, sequence or value is a historical reference, optionally embodied in a tangible medium. Typically, as would be understood by those skilled in the art, a reference agent, individual, population, sample, sequence or value is determined or characterized under conditions comparable to those utilized to determine or characterize the agent, individual, population, sample, sequence or value of interest.

“Small molecule”: As used herein, the term “small molecule” means a low molecular weight organic and/or inorganic compound. In general, a “small molecule” is a molecule that is less than about 5 kilodaltons (kD) in size. In some embodiments, a small molecule is less than about 4 kD, 3 kD, about 2 kD, or about 1 kD. In some embodiments, the small molecule is less than about 800 daltons (D), about 600 D, about 500 D, about 400 D, about 300 D, about 200 D, or about 100 D. In some embodiments, a small molecule is less than about 2000 g/mol, less than about 1500 g/mol, less than about 1000 g/mol, less than about 800 g/mol, or less than about 500 g/mol. In some embodiments, a small molecule is not a polymer. In some embodiments, a small molecule does not include a polymeric moiety. In some embodiments, a small molecule is not a protein or polypeptide (e.g., is not an oligopeptide or peptide). In some embodiments, a small molecule is not a polynucleotide (e.g., is not an oligonucleotide). In some embodiments, a small molecule is not a polysaccharide. In some embodiments, a small molecule does not comprise a polysaccharide (e.g., is not a glycoprotein, proteoglycan, glycolipid, etc.). In some embodiments, a small molecule is not a lipid. In some embodiments, a small molecule is a modulating agent. In some embodiments, a small molecule is biologically active. In some embodiments, a small molecule is detectable (e.g., comprises at least one detectable moiety). In some embodiments, a small molecule is a therapeutic.

“Therapeutic agent”: As used herein, the phrase “therapeutic agent” in general refers to any agent that elicits a desired pharmacological effect when administered to an organism. In some embodiments, an agent is considered to be a therapeutic agent if it demonstrates a statistically significant effect across an appropriate population. In some embodiments, the appropriate population may be a population of model organisms. In some embodiments, an appropriate population may be defined by various criteria, such as a certain age group, gender, genetic background, preexisting clinical conditions, etc. In some embodiments, a therapeutic agent is a substance that can be used to alleviate, ameliorate, relieve, inhibit, prevent, delay onset of, reduce severity of, and/or reduce incidence of one or more symptoms or features of a disease, disorder, and/or condition. In some embodiments, a “therapeutic agent” is an agent that has been or is required to be approved by a government agency before it can be marketed for administration to humans. In some embodiments, a “therapeutic agent” is an agent for which a medical prescription is required for administration to humans.

“Therapeutically effective amount”: As used herein, the term “therapeutically effective amount” means an amount that is sufficient, when administered to a population suffering from or susceptible to a disease, disorder, and/or condition in accordance with a therapeutic dosing regimen, to treat the disease, disorder, and/or condition. In some embodiments, a therapeutically effective amount is one that reduces the incidence and/or severity of, and/or delays onset of, one or more symptoms of the disease, disorder, and/or condition. Those of ordinary skill in the art will appreciate that the term “therapeutically effective amount” does not in fact require successful treatment be achieved in a particular individual. Rather, a therapeutically effective amount may be that amount that provides a particular desired pharmacological response in a significant number of subjects when administered to patients in need of such treatment. It is specifically understood that particular subjects may, in fact, be “refractory” to a “therapeutically effective amount.” To give but one example, a refractory subject may have a low bioavailability such that clinical efficacy is not obtainable. In some embodiments, reference to a therapeutically effective amount may be a reference to an amount as measured in one or more specific tissues (e.g., a tissue affected by the disease, disorder or condition) or fluids (e.g., blood, saliva, serum, sweart, tears, urine, etc). Those of ordinary skill in the art will appreciate that, in some embodiments, a therapeutically effective amount may be formulated and/or administered in a single dose. In some embodiments, a therapeutically effective amount may be formulated and/or administered in a plurality of doses, for example, as part of a dosing regimen

### DETAILED DESCRIPTION OF CERTAIN EMBODIMENTS

Various embodiments according to the present disclosure are described in detail herein. In particular, the present disclosure describes apparatus and methods for forming spatially registered patterns of cells; in some embodiments such spatially registered patterns are addressably linked to spatially registered patterns of wells.

Provided apparatus and methods find a variety of uses. In particular embodiments, provided apparatus and methods are useful for assessing functional responses of cells, for example to one or more environmental signals, conditions and/or stimuli.

Among other things, the present disclosure provides an insight that disclosed apparatus and methods provide spatially registered patterns of cells are particularly useful with beneficial attributes for monitoring cell-cell interactions, investigating cell-microenvironment interactions, and tracking cell migration or proliferation in response to environmental signal and/or stimuli.

### Slab

In some embodiments, apparatus disclosed herein includes a slab. In some embodiments, a slab is characterized in that it is manufactured from a moldable material. In some embodiments, a slab constructed from a biocompatible, elastomeric rubber material, so that it can flex. In some embodiments, a slab is formed in any conformable composition. In some embodiments, a moldable material is poly(dimethylsiloxane) (PDMS). In some embodiments, moldable material, for example, can include: plastic, polystyrene, polycarbonate, nitrocellulose, polyvinylidene fluoride, silicon, latex, natural rubber, hydrogels (e.g., collagen, polyacrylamide), and/or other gas-permeable, biocompatible materials preferably having a Young's modulus similar to PDMS, and/or metal.

In some embodiments, a slab is constructed using soft lithographic microengraving techniques such as casting and/or molding, for example, injection molding, transfer molding, compression molding, etc.). In some embodiments, a slab constructed using a master mold that includes a topographically-patterned surface that may be transferred to a slab.

In some embodiments, a slab is characterized in that it is manufactured from a deposited material. In some embodiments, a slab is characterized in that it is manufactured from a material that is etched. In some embodiments, methods for manufacturing rely on photolithography techniques.

In some embodiments, a slab includes at least one well formed in a surface of a slab. In some embodiments, a slab includes an array of wells includes formed in a surface of a slab. In some embodiments, a slab is characterized by an array of wells that form a spatially registered pattern in a surface of a slab.

In some embodiments, provided apparatus are characterized by an ability to design a slab. In some embodiments, provided apparatus are characterized in that a design, includes: a number of wells, a size of each well, a total size of a pattern, a total size of a slab, a total number of patterns on a surface of a slab, a shape of a slab, and spacing of patterns of wells.

In some embodiments, a slab has any shape capable of manufacture, including, for example a square, any polygon, etc. In some embodiments, a slab is a rectangle.

In some embodiments, a slab can be any size and/or dimension. In some embodiments, a slab is sized to conform with any standard microscope slide, for example 25 mm×60 mm, 24 mm×76 mm, etc. In some embodiments, a slab is no larger than the substrate with which it is to be sealed in accordance with the present invention; in some embodiments, a slab has comparable dimensions to that of the substrate.

In some embodiments, a slab is configured to have a substantially planar surface into which microwells extend.

In some embodiments, a slab is substantially rigid. In many embodiments, however, a slab is conformable, for example so that it can releasably seal with a substrate.

In some embodiments, slabs can be constructed using methods known in the art, including those described in PCT US 2006/036282 (published as WO 2007/035633) and U.S. Ser. No. 61/057,371, the contents of both of these applications are incorporated herein by reference in their entirety.

In some embodiments, each well of an array is characterized by a volume that the well is sized to contain. In some embodiments, a well is sized to contain between about a nanoliter to several hundred microliters. In some embodiments, a well is sized to contain about a nanoliter. In some embodiments, provided apparatus are characterized in that a slab with wells that are sized to contain a volume in a range of less than about a nL to about 250 μL. In some embodiments, well are sized to contain a volume of less than about a nL, less than about 1.5 nL, less than about 2.0 nL, less than about 2.5 nL, less than about 5.0 nL, less than about 7.5 nL, less than about 10 nL, less than about 15 nL, less than about 20 nL, less than about 25 nL, less than about 50 nL, less than about 75 nL, less than about 100 nL, less than about 150 nL, less than about 200 nL, less than about 250 nL, less than about 500 nL, less than about 750 nL, less than about 1.0 μL, less than about 1.5 μL, less than about 2.0 μL, less than about 2.5 μL, less than about 5.0 μL, less than about 10 μL, less than about 15 μL, less than about 20 μL, less than about 25 μL, less than about 50 μL, less than about 75 μL, less than about 100 μL, less than about 150 μL, less than about 200 μL, or less than about 250 μL.

In some embodiments, an array of wells defined in a surface of a slab are configured to hold a medium that supports cell growth and/or viability. In some embodiments, wells are configured to containing an environmental signal and/or stimulus in a volume within a well. In some embodiments, a medium containing an environmental signal and/or stimulus includes an infecting agent and/or a neutralizing agent in a volume within a well.

In some embodiments, wells may a fluid medium. In some embodiments, an array of wells is characterized in that when filled with a medium and contacted with a substrate, excess fluid is expelled. In some embodiments, excess fluid is present between a substrate and a surface of a slab. In some embodiments, channels are defined in a surface of a slab so that excess fluid present in wells is squeezed from a space between a substrate and a surface of a slab. In some embodiments, an array of wells is configured with microchannels defined in a surface of a slab. In some embodiments, excess fluid is removed from a slab surface via at least one microchannel. In some embodiments, microchannels are configured in a surface of the slab. In some embodiments, excess fluid is squeezed from a space between a substrate and a surface of a slab into microchannels. In some embodiments, Microchannels as used and described herein are known in the art, including those described in U.S. Pat. No. 8,569,046, the contents of which is incorporated herein by reference in its entirety.

### Substrate

In some embodiments, the present invention utilizes a on which cells are patterned. In general, a substrate may be formed of any material compatible with cell viability and/or analysis. In some embodiments, a substrate is made of glass. In some embodiments, a substrate is any standard microscope slide. In some embodiments, a substrate is made for example of plastic, ceramic, metal, silicon, rubber, or any material known in the art.

In some embodiments, a substrate provides a surface for coating a layer of cells. In some embodiments, a surface of a substrate is substantially planar.

In some embodiments, a layer of cells is a monolayer of targets cells. In some embodiments, a substrate surface is uniformly coated with cells. In some embodiments, a substrate uniformly coated with cells is indicative of a uniform cell size distribution. In some embodiments, a uniform cell size distribution is one in which a majority of cells within a population of same cells have a cell diameter within a specified variance. In some embodiments, a majority is more than 50%, 60%, 70%, 75%, 80%, 85%, 90%, 95%, 96%, 97%, 98%, 99%, 99.5%, 99.6%, 99.7%, 99.8%, 99.9% or more cells in a cell population. In some embodiments, a variance in a diameter size is less than about 50%, about 40%, about 30%, about 25%, about 20%, about 15%, about 10%, about 5%, about 4%, about 3%, about 2%, about 1%, about 0.5%, about 0.4%, about 0.3%, about 0.2%, about 0.1% or less. In some embodiments, a substrate uniformly coated with cells is indicative of a substrate surface having about less than 20% surface area not coated with a cell, having about less than 15% surface area not coated with a cell, having about less than 10% surface area not coated with a cell, having about less than 5% surface area not coated with a cell, having about less than 4% surface area not coated with a cell, having about less than 3% surface area not coated with a cell, having about less than 3% surface area not coated with a cell, having about less than 2% surface area not coated with a cell, having about less than 1% surface area not coated with a cell, having about less than 0.5% surface area not coated with a cell or less.

In some embodiments, a surface of a substrate is functionalized with a retention agent. In some embodiments, a retention agent binds to cells. In some embodiments, a retention agent binds to a particular subset of cells (e.g., of a particular cell type and/or developmental stage, etc.). In some embodiments, a retention agent is or comprises an adhesion molecule. In some embodiments, a retention agent is or comprises an antibody.

In some embodiments, cells are adhered to a surface (or portion thereof) of a substrate. In some embodiments, cells are substantially uniformly adhered to a surface (or portion thereof) of a substrate. In some embodiments, cells are adhered to a substrate in a monolayer.

### Cells

In general, provided technologies are useful for patterning of any cells. In some embodiments, cells patterned in accordance with the present invention are eukaryotic cells. In some embodiments, cells patterned in accordance with the present invention are prokaryotic cells. In some embodiments, cells patterned in accordance with the present invention are invertebrate cells, vertebrate cells, animal cells, mammalian cells, human cells, etc.

In some embodiments, cells patterned in accordance with the present invention are characterized in that they are susceptible to infection with one or more infecting agents.

In some embodiments, cells patterned in accordance with the present invention are characterized in that they participate in cell-cell interactions with other cells.

In some embodiments, cells patterned in accordance with the present invention are characterized in that they are disease cells, or models thereof.

### Patterned Cells

Among other things, the present invention encompasses the insight that spatially patterned cell arrangements can readily be generated by contacting a slab as described herein with a substrate whose surface contains cells as described herein, so that cells are disrupted at points of contact between the slab and the substrate, and cells remain intact elsewhere. Thus, in some embodiments, the present disclosure provides substrates with cells adhered thereto in a particular spatial patterning arrangement that corresponds to the structural arrangement of wells in a slab.

Thus, in some embodiments, patterned cells are adhered at discrete positions on a surface of a substrate, which positions correspond to locations, dimensions, and/or arrangement of cells in a slab.

Environmental Signal and/or Stimuli

In some embodiments, provided technologies are utilized to assay and/or characterize response of patterned cells to one or more environmental agents, signals, stimuli and/or conditions. Indeed, one particularly advantageous aspect of technologies provided herein is that they permit particularly facile analysis of cellular behaviors and/or responses to such agents, signals, stimuli and/or conditions.

For example, in some embodiments, environmental agents, signals, stimuli and/or conditions whose effects are assessed in accordance with the present invention include particular pH, salt concentration, osmotic stress, temperature, pressure, presence or absence of radiation (e.g., of a particular wavelength such as visible or ultraviolet), presence or absence of a magnetic field, presence or absence or direction of flow, presence or absence of mechanical stress, presence or absence or level of a particular chemical or biological agent etc., or combinations thereof.

In some particular embodiments, an environmental agent, signals, stimulus or condition is one that may inhibit, promote, or otherwise modulate one or more aspects of cell viability, growth, differentiation, mobility, etc. In some embodiments, such an agent, signal, stimulus or condition may be or include a small molecule agent (e.g., a toxin or a drug or drug candidate), a biologic agent (e.g., a polypeptide such as a cytokine or an antibody having a particular biologic activity or effect of interest; a nucleic acid such as an siRNA agent, an antisense agent, an aptamers, etc; a glycan; a lipid, or any combination thereof). In some embodiments, such an agent, signal, stimulus or condition may be or include an infectious agent.

In some embodiments, an environmental agent, signal, stimultus or condition is, comprises, or is provided by a cell (e.g., that secretes or otherwise presents a particular chemical or biologic agent). In some such embodiments, such cells are distributed in microwells of the slab. In some embodiments, such cells are distributed at a density, on average, of about 1-5 cells/well. In some embodiments, such cells are distributed so that each microwell contains not more than about 10-20 cells, or not more than about 10 cells, or not more than about 5 cells, or not more than about a few cells or not more than about 2 cells, or not more than about 1 cell per well.

In some embodiments, an environmental agent, signal, stimulus or condition mimics, models, or induces one or more features of a disease, disorder or condition such as, for example, a proliferative disease, disorder or condition, and inflammatory disease, disorder or condition, an infection, a immunoregulatory disease disorder or condition, a metabolic disease, disorder or condition, a neurological disease disorder or condition, etc. To give but a few examples, in some embodiments, autoimmune diseases, for example, include: arthritis (including rheumatoid arthritis), multiple sclerosis, immunemediated or Type 1 diabetes mellitus, inflammatory bowel disease, systemic lupus erythematosus, psoriasis, scleroderma, and autoimmune thyroid diseases. In some embodiments, infectious diseases, for example, include: African trypanosomiasis, cholera, cryptosporidiosis, dengue, hepatitis A, hepatitis B, hepatitis C, HIV/AIDS, influenza, malaria, Japanese encephalitis, malaria, measles, meningitis, onchocerciasis (“river blindness”), pneumonia, rotavirus, schistosomiasis, shigellosis, strep throat, tuberculosis, typhoid, and yellow fever.

In some embodiments, provided medium containing an environmental signal and/or stimulus includes a disease-causing agent (e.g., an infecting agent) and/or a potential neutralizing or therapeutic agent in a volume within a well.

In some embodiments, an environmental signal and/or stimuli is or comprises an infecting agent. In some embodiments, an environmental signal and/or stimuli that is or comprises an infecting agent is a virus, for example HIV.

In some embodiments, an environmental signal and/or stimuli is or comprises a neutralizing agent. In some embodiments, a neutralizing agent is specific to another environmental signal and/or stimuli. In some embodiments, an environmental signal and/or stimuli that is or comprises a neutralizing agent is an antibody producing cell. In some embodiments, a neutralizing agent produces an antibody. In some embodiments, an antibody produced by a neutralizing agent is a specific response to an infecting agent, for example, an antibody specific to a virus.

In some embodiments, an environmental signal and/or stimuli is present in a medium.

In some embodiments, a medium is an aqueous composition (e.g., a solvent or dispersing medium is or comprises water). In some embodiments, a solvent and/or dispersing medium, for example, is or comprises water, cell culture medium, buffers (e.g., phosphate buffered saline), buffered solutions (e.g. PBS), polyols (for example, glycerol, propylene glycol, liquid polyethylene glycol, and the like), agar, gelatin, Dulbecco's Modified Eagle Medium, fetal bovine serum, or suitable combinations and/or mixtures thereof.

### Methods of Well-Assisted Patterning

In some embodiments, as described herein, the present invention provides an apparatus that includes a cell-coated slab and a substrate configured to be coupled with a micro-well slab so that cells on the substrate become spatially patterned according to the arrangement of micro-wells in the slab.

In some embodiments, such patterning is achieved by coupling and reversibly sealing a cell-coated surface of a substrate to a surface of a microwell-containing slab. In some embodiments, a reversible seal is a substantially fluid tight seal between a surface of a slab and a surface of a substrate.

In some embodiments, a substrate containing cells on its surface is coupled and releasably sealed to a slab so that cells on the substrate either become disrupted due to contact with the slab surface or remain intact due to positioning in register with a microwell in the slab surface, so that the cells on the substrate become arranged in a pattern that is spatially registered with respect to the arrangement of wells in the slab. In some embodiments, a slab and a substrate are coupled and reversibly sealed for a period of time. In some embodiments, a period is an incubation time. In some embodiments, an incubation time is at least about 1 min., about 1.5 min., about 2 min., about 3 min., about 4 min., about 5 min., about 6 min., about 7 min., about 8 min., about 9 min., about 10 min., about 15 min., about 20 min., about 25 min., about 30 min., about 35 min., about 40 min., about 45 min., about 50 min., about 55 min., about 1 hour., about 1.5 hours., about 2 hours, about 3 hours, about 4 hours, about 5 hours, about 6 hours, about 7 hours, about 8 hours, about 9 hours, about 10 hours, about 11 hours, about 12 hours, about 24 hours, about 48 hours, or longer.

In some embodiments, a spatially registered pattern of cells is formed on a surface of a substrate when a substrate coated with a layer of cells is released from a surface of a slab (and, e.g., components of disrupted cells are washed away).

In some embodiments, coupling of a substrate to a slab generates a spatially registered pattern of cells. In some embodiments, a spatially registered pattern of cells provides an addressable link to a spatially registered pattern of well. In some embodiments, an addressable link therefore exists between an individual cell or set of cells and a well location. In some embodiments, a well location is an addressable link within a spatially registered pattern of wells.

In some embodiments, alignment of a substrate coated with cells and sealing to a slab forms a spatially registered pattern of cells according to a spatially registered pattern of wells defined in a surface of a slab. In some embodiments, sealing and provides an addressable link between an individual cell and a well location within spatially registered pattern for an array of wells. In some embodiments, a well location is addressable. In some embodiments, an addressable link is an individual cell and a well location that provides a correlation between a functional response of an individual cell with an environmental signal and/or stimuli of a well.

In some embodiments, an addressable link between an individual cell and a well location that provides a correlation between a functional response of an individual cell with an environmental signal and/or stimuli present in a medium within a volume of a well.

In some embodiments, a correlation between an individual cell and a well location that is addressable can be aggregated to assess a correlation of all cells within a pattern of a slab. In some embodiments, a correlation between an individual cell and a well location that is addressable can be aggregated to assess a correlation of all cells similarly situated. In some embodiments, above described functional assays that employ living cell arrays can allow for the identification of stimuli that elicit complex cellular responses.

### Methods of Use

Those of ordinary skill in the art, reading the present disclosure, will appreciate the wide range of useful applications of the present technology. In some embodiments, technologies described herein are used to assess cellular activity or response (e.g., to a particular environmental signal, stimulus, and/or condition. In some embodiments, provided technologies are used to identify and/or characterize agents of interest (e.g., agents that impact cellular viability, activity, growth, proliferation, development, and/or movement). In some embodiments, steps of provided such methods are repeated more than once. In some embodiments, the present disclosure includes methods of assessing at multiple points in time.

In some embodiments, provided methods are characterized in that they represent simple and robust strategies for high-throughput screening of responses of single cell to an environmental signal and/or stimuli. In some embodiments, provided methods are characterized by a series of coordinated events.

In some embodiments, methods of creating arrays of wells used for patterning and deposition of cells and proteins into wells has been previously described in Love, J. C., “A microengraving method for rapid selection of single cells producing antigen-specific antibodies,” 24 Nat Biotechnol, 703-707 (2006), the contents of which is incorporated herein by reference in its entirety. In some embodiments, WAP relies on mechanical disruption of cells on a surface of a substrate by a spatially registered pattern of wells defined in a surface of a slab. In some embodiments, mechanical disruption occurs when regions of a surface of a substrate contact regions of a surface of a slab with spatially registered pattern of wells defined therein.

In some embodiments, provided methods include steps of functionalizing a surface of a substrate (e.g. a microscope slide) with cell surface-specific antibodies or non-specific adhesion molecules. In some embodiments, provided methods include steps of coating and/or uniformly coating a surface of a substrate with cells of interest.

In some embodiments, provided methods include providing a substrate coated with a monolayer of cells. In some embodiments, provided methods include steps of providing a slab comprising an array of wells formed into a surface of the slab, wherein the wells a characterized by the presence of an environmental signal and/or stimuli. In some embodiments, each well can contain a different an environmental signal and/or stimuli of interest, such as an agent.

In some embodiments, provided methods include steps of exposing cells coated on a surface of a substrate to a surface of a slab comprising an array of wells formed into a surface of the slab. In some embodiments, provided methods include sealing a substrate coated with cells against a surface of a slab. In some embodiments, provided methods include forming a substantially fluid tight seal between a surface of a slab and a surface of a substrate.

In some embodiments, provided methods include steps of mechanically disrupting cells coated on a surface of a substrate through contacting cells with a portion of a surface of a slab which is substantially planar. In some embodiments, provided methods include mechanically disrupting cells according to a pattern formed by wells formed in a surface of a slab, thereby creating a spatially registered pattern of cells. In some embodiments, mechanically disrupting cells occurs where a surface of a slab defining an array of wells contacts a surface of a substrate coated in a monolayer of cells. In some embodiments, provided methods include steps of exposing cells coated on a surface of a substrate to spatially registered pattern of wells on a surface of a slab are not mechanically disrupted.

In some embodiments, provided methods include incubating cells in wells. In some embodiments, incubating cells in wells when a substrate is releasably sealed to a slab occurs over a period. In some embodiments, a period for an incubating step is about 1 min., about 1.5 min., about 2 min., about 3 min., about 4 min., about 5 min., about 6 min., about 7 min., about 8 min., about 9 min., about 10 min., about 15 min., about 20 min., about 25 min., about 30 min., about 35 min., about 40 min., about 45 min., about 50 min., about 55 min., about 1 hour., about 1.5 hours., about 2 hours, about 3 hours, about 4 hours, about 5 hours, about 6 hours, about 7 hours, about 8 hours, about 9 hours, about 10 hours, about 11 hours, about 12 hours, about 24 hours, about 48 hours, or longer.

In some embodiments, provided methods include steps of releasing a surface of a substrate from a surface of a slab defined by a spatially registered pattern of wells, resulting in a spatially registered patter of target formed by mechanical disruption.

In some embodiments, provided methods include washing away mechanically disrupted cells and leaving cells that are adhered and viable on a coated substrate.

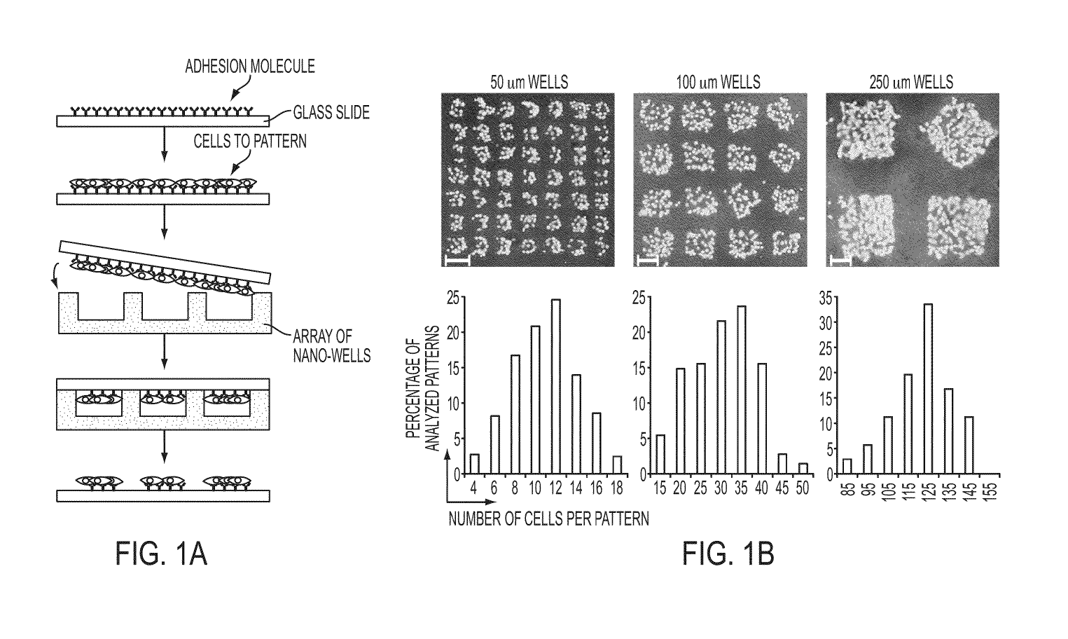

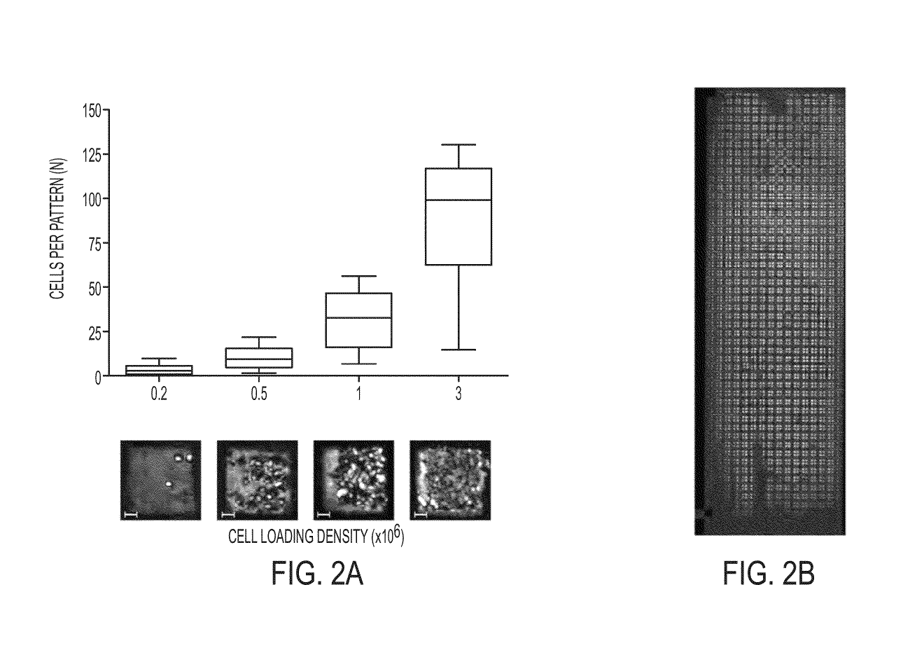

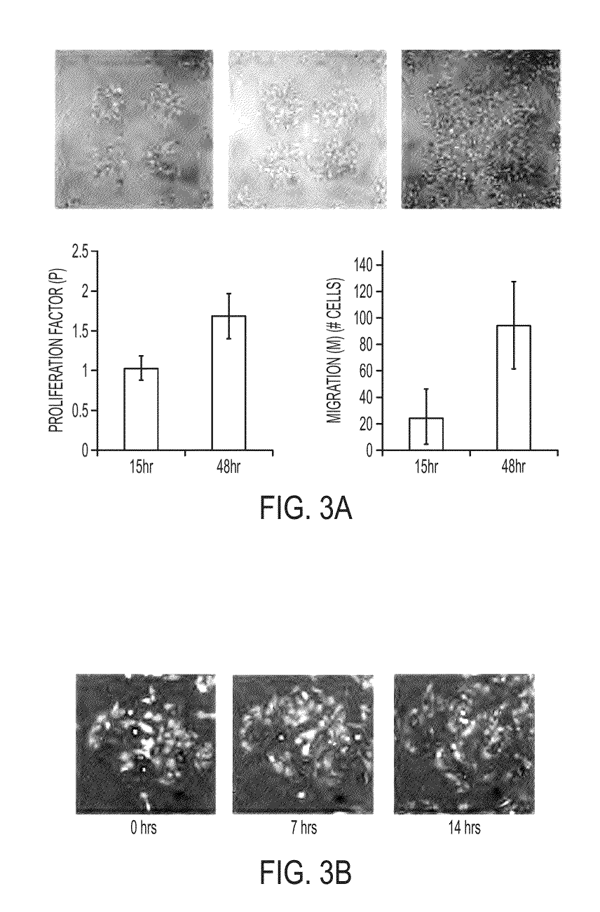

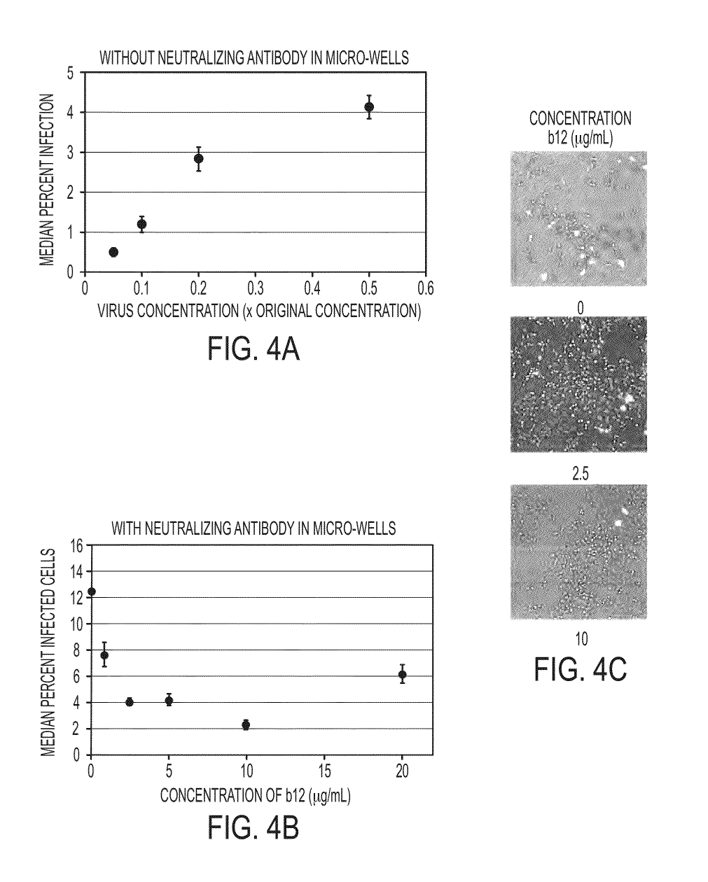

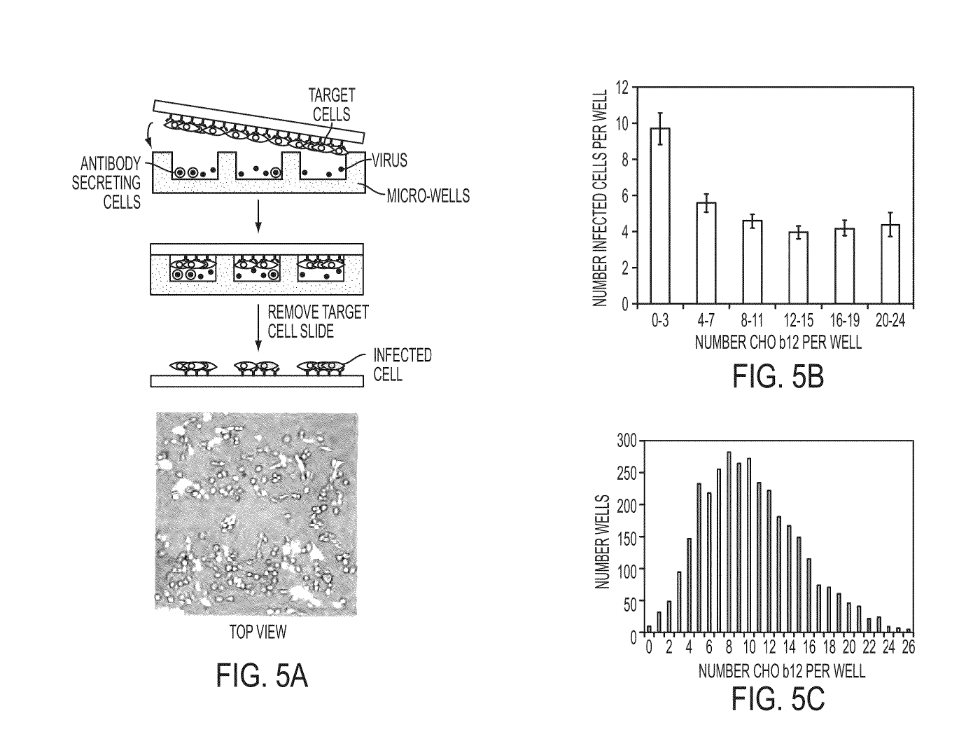

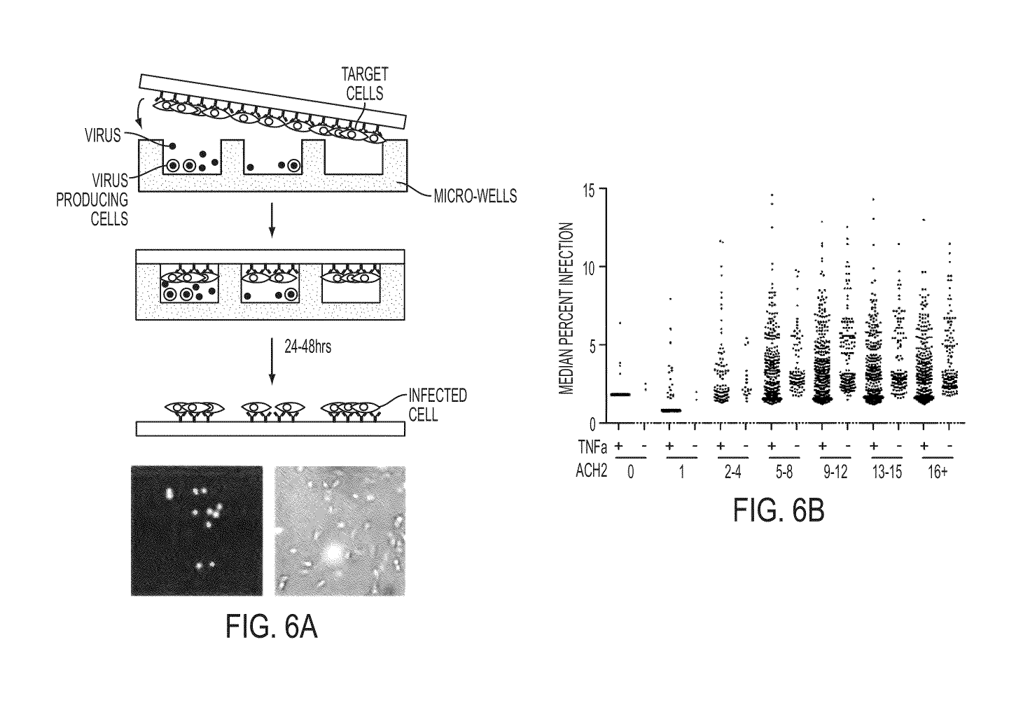

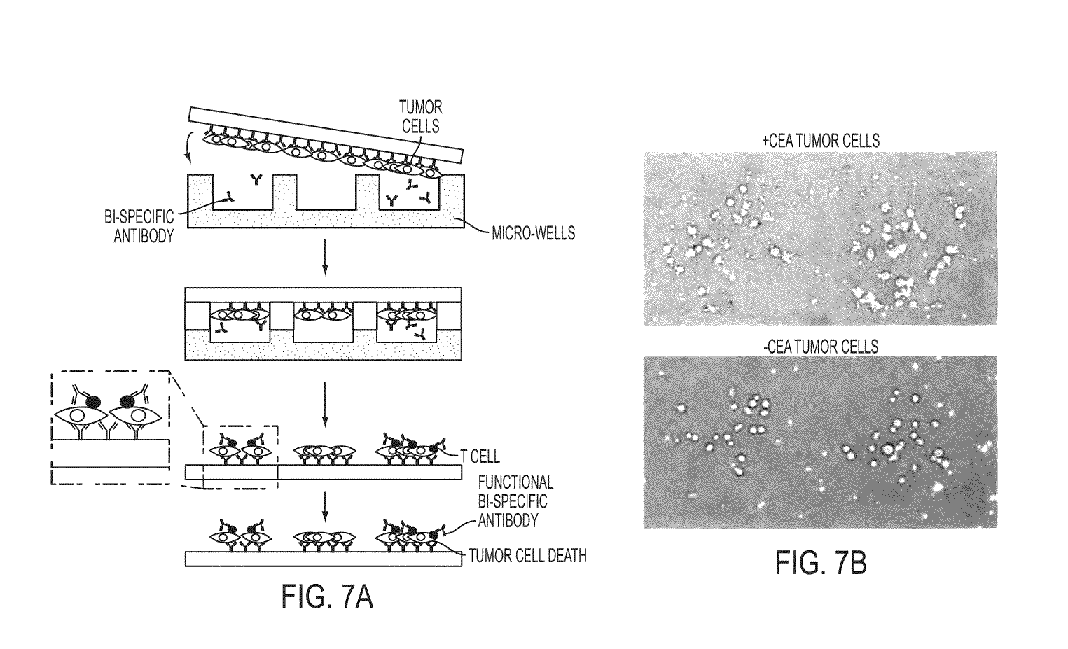

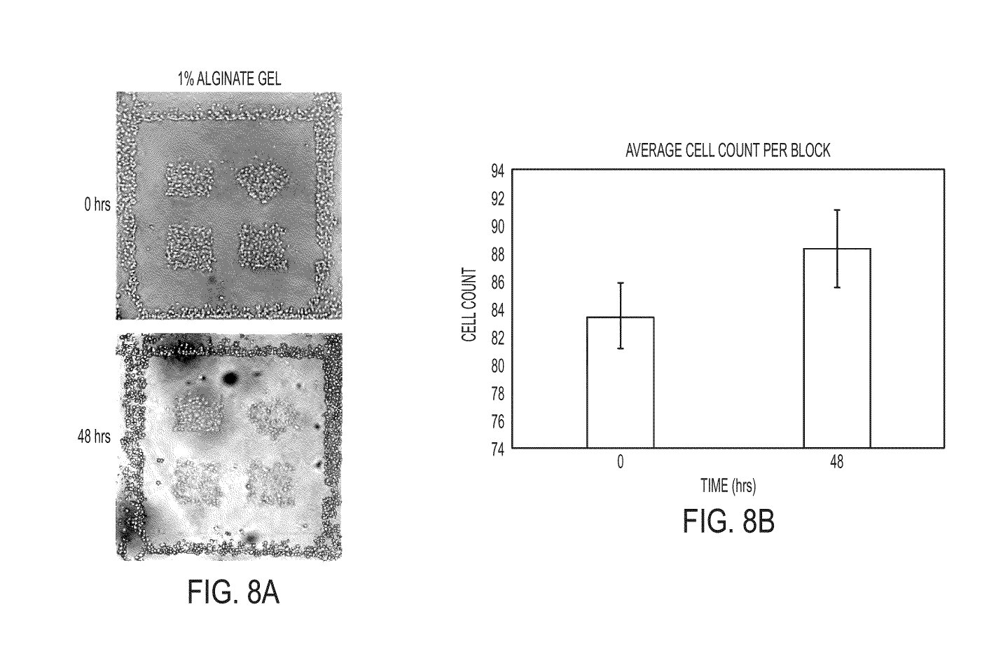

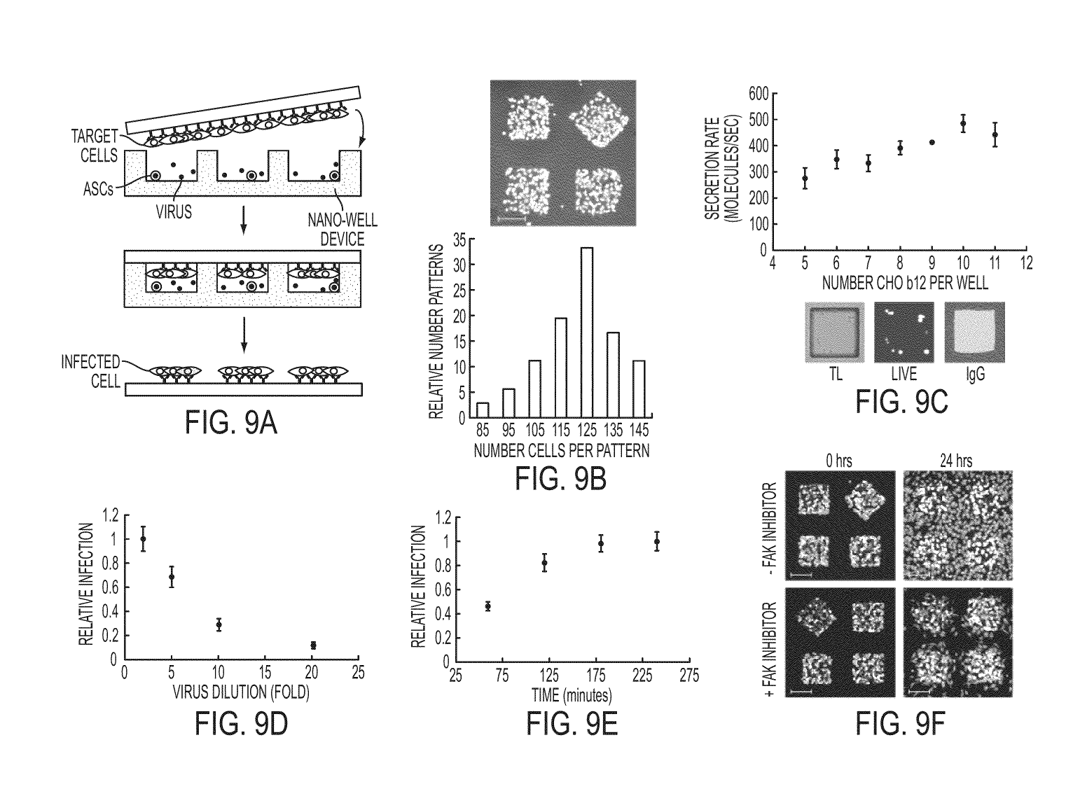

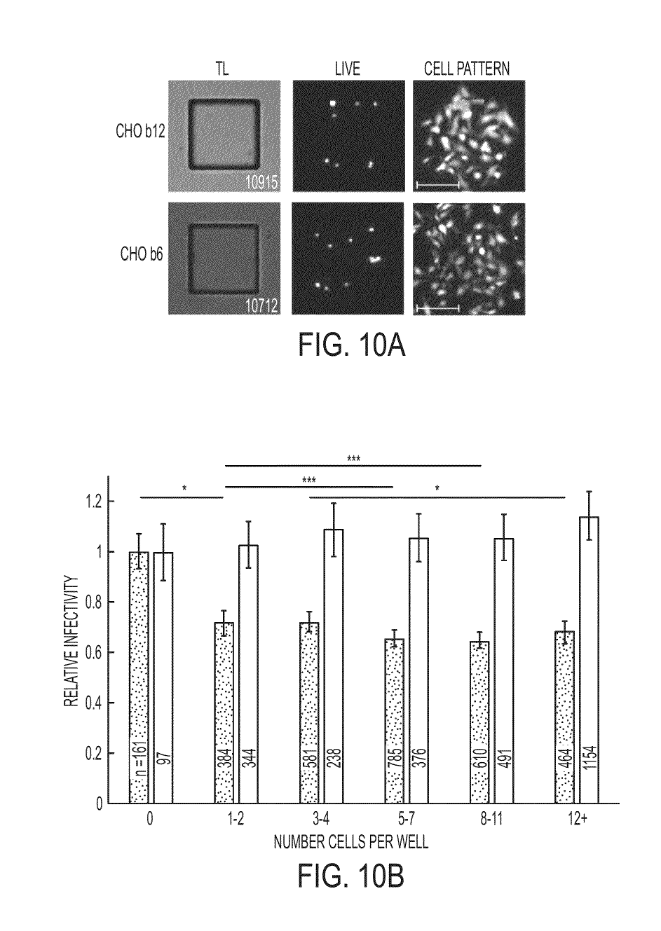

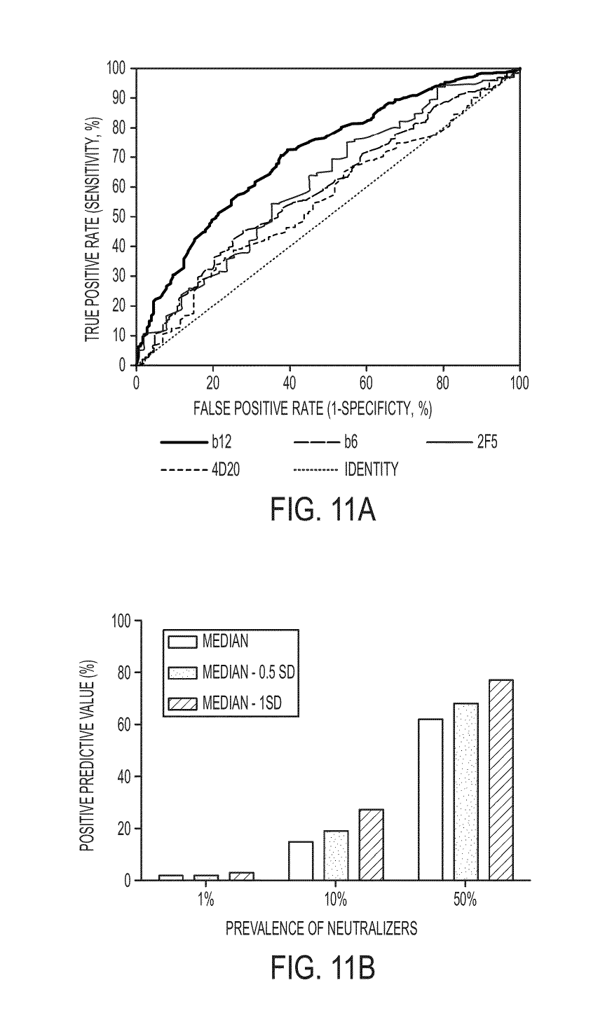

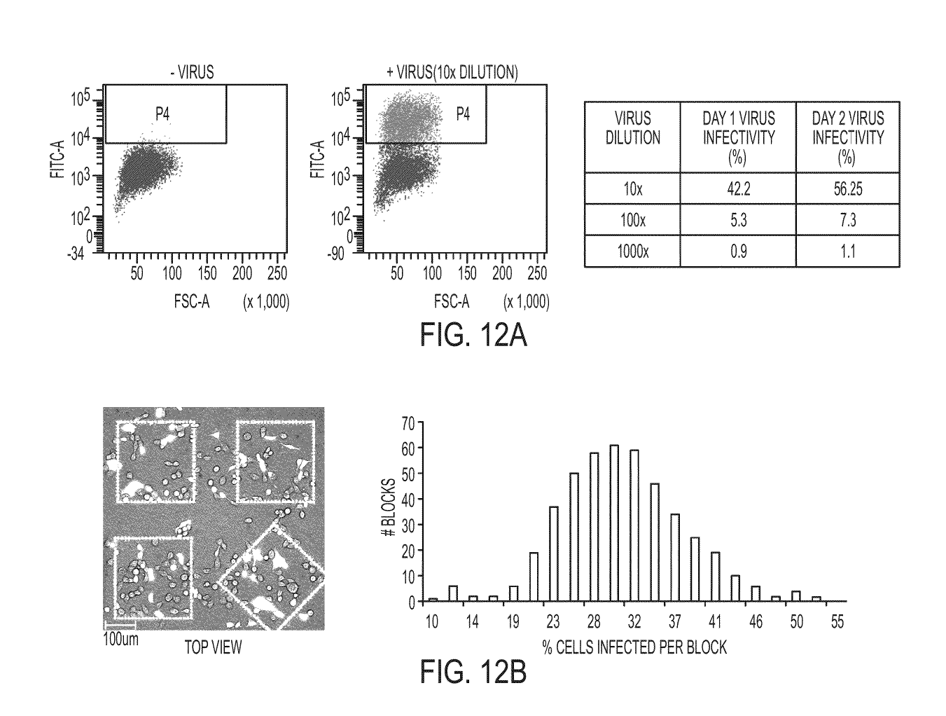

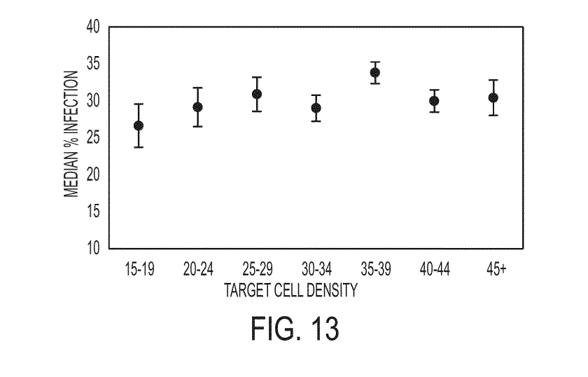

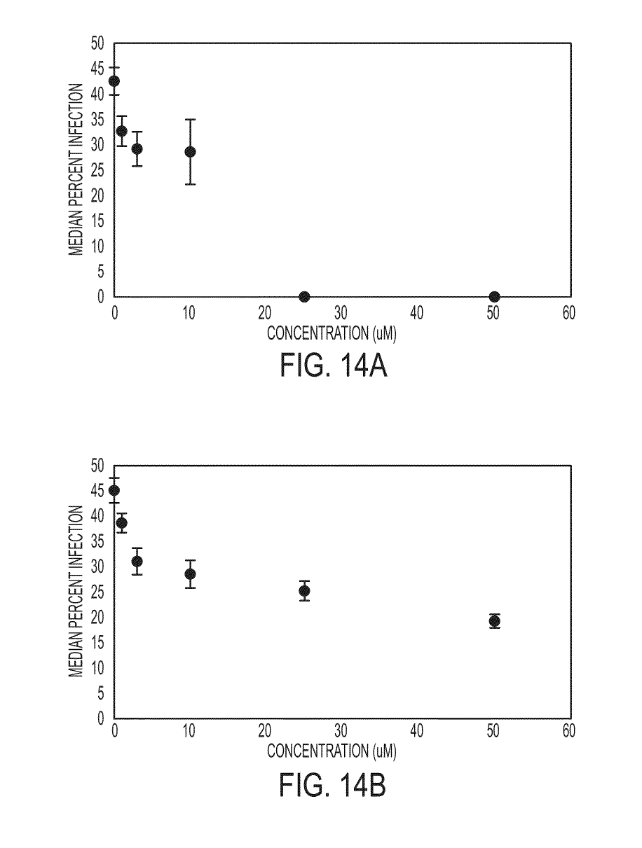

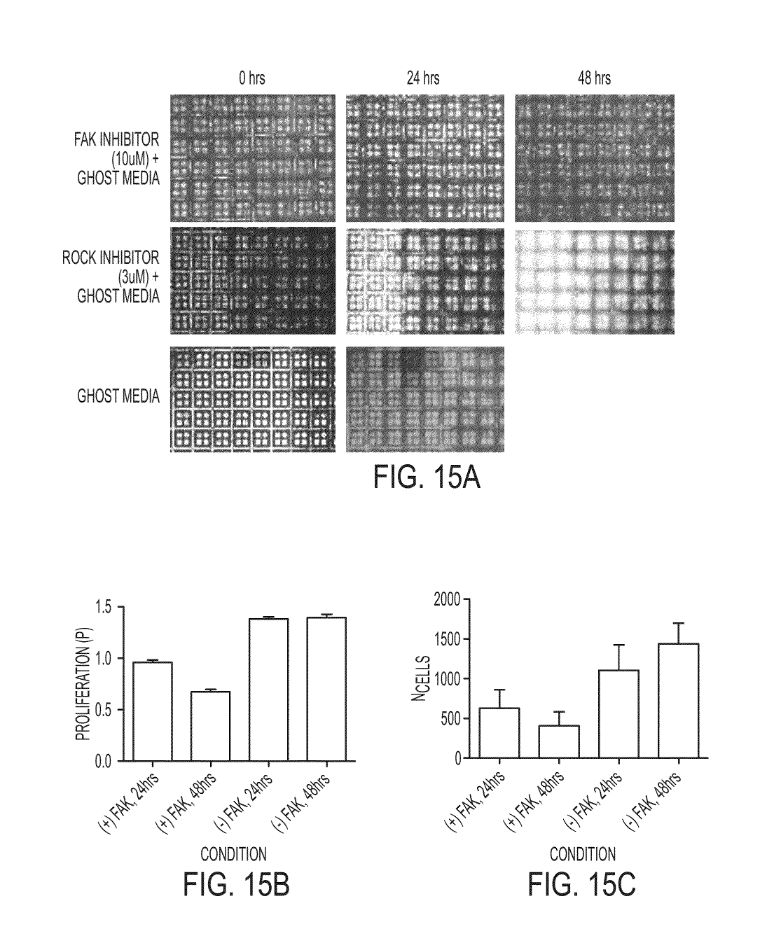

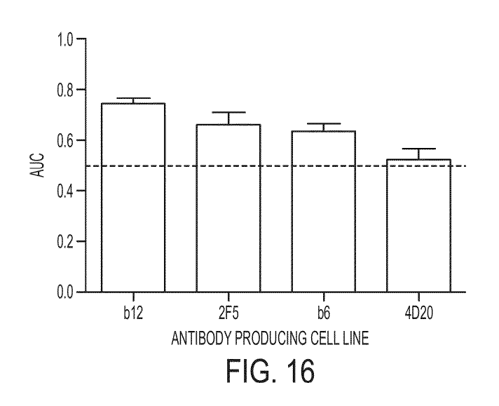

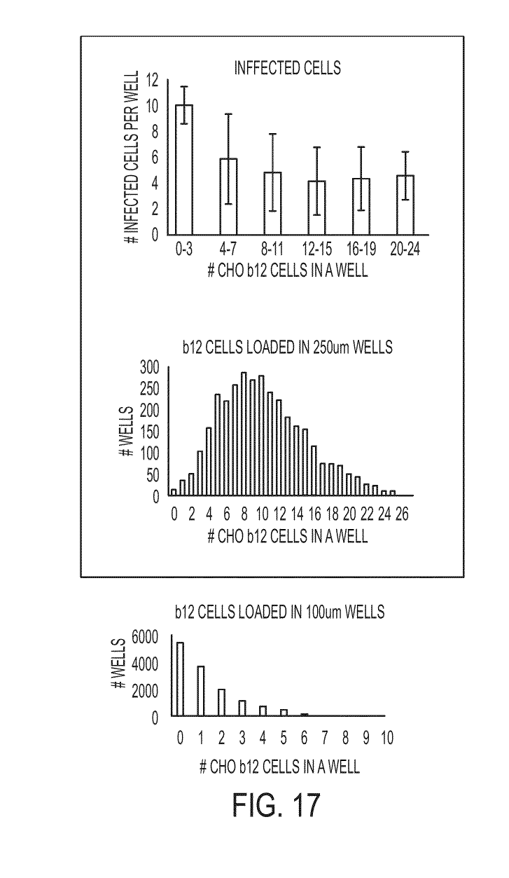

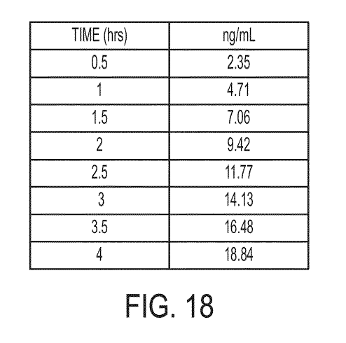

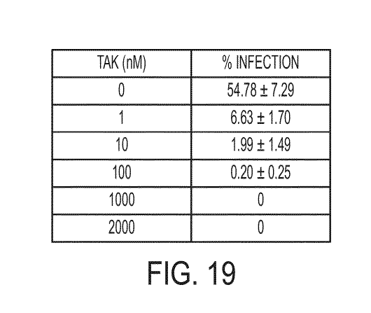

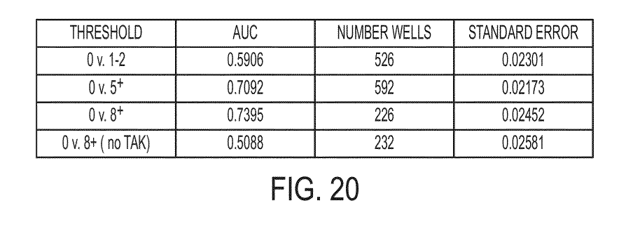

In some embodiments, cells forming a spatially registered pattern of cells remain attached to a surface and viable. FIG. 1(a) (bottom panel) shows a spatially registered pattern of “viable” cells on surface of a substrate after release of a substrate from a slab.

In some embodiments, a separate scale assay is formed at each spatially registered pattern of wells. In some embodiments, a portion of a surface of a substrate coated with cells interacts with a specific well of a spatially registered pattern of wells.

In some embodiments, provided methods include measuring biologically relevant parameters of an individual cell and/or cells.

In some embodiments, provided methods include linking by an addressable link individual cells to a specific well of a spatially registered pattern of wells.

In some embodiments, provided methods include assessing a change in a functional response of a cell to an environmental signal and/or stimuli. In some embodiments, provided methods include assessing an integrated response of a cell of a spatially registered pattern of cells to environmental signal and/or stimuli. In some embodiments, cells come together environmental signal and/or stimuli. In some embodiments, cells are exposed to infecting agents and/or neutralizing agents.

Many methods have been developed to adhere intact and/or living cells on surfaces Any such methodologies may be utilized in the practice of the present invention.

Presently provided technologies represent significant improvements over other available cell patterning technologies, many of which rely on creating patterns of positive and negative adherence materials for the cells to attach to or be repelled from, including microarray printing, (see Flaim, C. J. et al., “An Extracellular Matrix Microarray for Probing Cellular Differentiation,” 2 Nat Meth, 119-125 (2005); Reticker-Flynn, N. E. et al., “Combinatorial Extracellular Matrix Platform Identifies Cell-Extracellular Matrix Interactions That Correlate with Metastasis,” 3 Nature Communications, 1122 (2012); Woodruff, K. et al., “Live Mammalian Cell Arrays,” 10 Nat Meth, 550-552 (2013); soft lithography based stamping, (see Chen, C. S., “Geometric Control of Cell Life and Death,” 276 Science, 1425-1428 (1997); microfluidics (see Takayama et al., “Microfabrication and Microfluidics-Based Patterning of Cultured Neuronal Network,” journal 1-4 (2011); layer-by-layer assembly (see Berg, M. C. et al., “Controlling Mammalian Cell Interactions on Patterned Polyelectrolyte Multilayer Surfaces.” 20 Langmuir, 1362-1368 (2004); acoustic waves (see Ding, X. et al., “Tunable patterning of microparticles and cells using standing surface acoustic waves,” 12 Lab Chip, 2491 (2012); or UV cross-linking (see Fink, J. et al., “Comparative study and improvement of current cell micro-patterning techniques,” 7 Lab Chip, 672 (2007), to name a few. Additionally, methods of blocking exposed surfaces with polymers and cell encapsulation with hydrogels have been developed shown and can be used to maintain patterned cells for extended periods of time. (See Winblade, N. D. et al., “Sterically blocking adhesion of cells to biological surfaces with a surface-active copolymer containing poly(ethylene glycol) and phenylboronic acid,” 59 J. Biomed. Mater. Res., 618-631 (2001) and Khattak, S. F., “Pluronic F127 as a cell encapsulation material: utilization of membrane-stabilizing agents,” 11 Tissue Engineering, 974-983 (2005)).

While the above mentioned techniques have emerged for creating cell-based assays, these methods are not suited for studying complex functional assays. In each case a link between stimuli and cell is not spatially maintained. Moreover, the existing mentioned approaches for creating living cell based arrays rely on complicated surface modifications, require special equipment, and lack flexibility for designing high throughput living cell-based screens.

In some embodiments, provided apparatus and methods are characterized in that they offer a simple yet efficient way to spatially register patterns of cells so that cells are easily and efficiently correlated to an associated well containing an environmental signal and/or stimuli of interest.

In some embodiments, provided apparatus and methods do not rely on complicated surface modifications to create and maintain cell patterns over biologically relevant time scales.

In some embodiments, provided apparatus and methods allow for high-throughput measurements of cells exposed to environmental signal and/or stimuli contained within a medium of a well.

In some embodiments, provided apparatus and methods are characterized by an ability to study an effect of environmental signal and/or stimuli on cells. In some embodiments, provided apparatus and methods measure biologically relevant parameters of cells. In some embodiments, provided apparatus and methods study cell fate and/or phenotype.

In some embodiments, provided apparatus and methods are characterized by an ability to read out a dynamic and/or an integrated response of cells to an environmental signal and/or stimuli. In some embodiments, provided apparatus and methods assess functional responses over a period. In some embodiments, provided apparatus and methods assess responses by incubating cells with an environmental signal and/or stimuli. In some embodiments, provided apparatus and methods assess responses through multiple measurements separately acquired over a period. In some embodiments, provided apparatus and methods assess responses of the same cells through multiple measurements by iteratively repeating periods of cell exposure to an environmental signal and/or stimuli. In some embodiments, provided apparatus and methods assess responses of the same cells through multiple measurements by repeating exposure periods with changes to an environmental signal and/or stimuli. As such, provided apparatus and methods are characterized by an ability to provide a better understanding of complex behavior in cells.

In some embodiments, provided methods and apparatus are characterized by an ability to detect a percentage of cells infected at each cell of a spatially registered pattern of cells, and discriminate between neutralizing, weakly neutralizing, and non-neutralizing antibodies using established antibody-secreting cell lines.

In some embodiments, provided apparatus and methods offer a unique combination, for example, in which antibody secreting cells, virus particles, and cells are brought together in spatially registered, high-throughput, and in nanoliter-scale to microliter-scale environments using arrays of wells.

In some embodiments, provide apparatus and methods feature single-cell counting capability for infection, short incubation times, and no pre-incubation of virus and antibody suggest that it should facilitate the identification of strong neutralizing antibodies in screens. In some embodiments, provided apparatus and methods are characterized by scalability so that about 10⁴ cells per assay can be evaluated in about 3 days.

In some embodiments, provided apparatus and methods allow for control over cell density for spatially registered pattern of cells and provide a flexible platform to create discrete co-cultures of desired cell types.

In some embodiments, provided apparatus and methods that include spatially registered patterns of wells forming spatially registered patterns of cells on a substrate enabling readout of dynamic and integrated responses, rather than just binding affinities.

In some embodiments, provided apparatus and methods including spatially registered patterns of wells forming spatially registered patterns of cells therefore offer an advantage over existing methods, devices, or materials, such as biomolecular arrays where the resultant readout or output only provides information related to binding affinities. (See for example, Fernandes, T. G. et al., “High-Throughput Cellular Microarray Platforms: Applications in Drug Discovery, Toxicology and Stem Cell Research,” 27 Trends in Biotechnology, 342-349 (2009). With living cell arrays, controlled systems in which to study the effect of stimuli, such as virus or extracellular matrix, on cell fate and phenotype have been realized with high-throughput and scalability. (See Flaim, C. J. et al., “An Extracellular Matrix Microarray for Probing Cellular Differentiation,” 2 Nat Meth, 119-125 (2005); Albrecht, D. R. et al., “Photo- and Electropatterning of Hydrogel-Encapsulated Living Cell Arrays,” 5 Lab Chip, 111 (2005); and Reticker-Flynn, N. E. et al., “Combinatorial Extracellular Matrix Platform Identifies Cell-Extracellular Matrix Interactions That Correlate with Metastasis,” 3 Nature Communications, 1122 (2012)).

In some embodiments, provided apparatus and methods are characterized in that they allow study of cells and cell behavior in a medium containing an environmental signal and/or stimuli.

In some embodiments, an environmental signal and/or stimuli comprises an infecting agent and/or a neutralizing agent.

In some embodiments, provided apparatus and methods are characterized by a functional cell-based assay. In some embodiments, a functional cell-based assay provides a format including an array of wells. In some embodiments, a signal readout provides an indication of cell behavior or cell response to an environmental signal and/or stimulus, for example an infection and/or neutralization.

In some embodiments, provided apparatus and methods allow interactions to occur between cells and an environmental signal and/or stimuli of interest. In some embodiments as compared to existing methods for creating cell-based arrays, provided apparatus and methods offer a systematic way to study of how stimuli effect cell behavior. In some embodiments, provided apparatus and methods allow for measuring a dynamic and/or integrated response of cells to an environmental signal and/or stimuli of interest.

In some embodiments, provided apparatus and methods allow for spatially registered pattern of cells to be linked by an addressable link to a spatially registered pattern of wells containing an environmental signal and/or stimuli of interest. That is, provided apparatus and methods are characterized by an ability to spatially register patterns of cell with cognate wells containing an environmental signal and/or stimuli of interest, for example an antibody secreting cell, virus, and/or growth factor, to name a few.

In some embodiments, provided apparatus and methods allow for measurements of interactions of spatially registered patterns of cells within a discrete environment of a spatially registered pattern of wells.

In some embodiments, provided apparatus and methods are characterized in that they can perform with varied types of interactions including, but not limited to: secretion of antibodies specific to surface markers on patterned cells, activation of patterned cells, cytolytic activity such as secretion of cytotoxic proteins by cells in microwells or active killing of patterned cells, quantification of antibody mediated cellular cytotoxicity, or infection of patterned cells by viral particles produced by cells in wells.

In some embodiments, provided apparatus and methods are characterized by a high-throughput approach that allows for the interrogation of thousands of antibody secreting cells in parallel while leveraging miniaturization to reduce reagent use.

In some embodiments, a key advantage of provided apparatus and methods technique is an ability to spatially register cell patterns with cognate wells, where thousands of wells can be interrogated in parallel. Unlike conventional cell patterning methods, this platform can be readily adapted for high throughput screens to identify cells with desired function or cells producing desired therapeutics.

Provided methods were validated when neutralization of pseudotyped HIV using cell lines secreting known neutralizing antibodies was detected. Validation using HIV demonstrated the potential for a high-throughput single-cell-scale technology for the identification of neutralizing agents and/or therapeutic agents with drastically reduced time and resources, which may be applicable to other infectious diseases or cancer.

It is apparent to those skilled in the art that provided apparatus and methods have broad applicability beyond HIV.

In some embodiments, provided apparatus, e.g. WAP and provided methods can be used to measure many cell parameters and behaviors in parallel, such as migration, proliferation, differentiation, or apoptosis, in response to various stimuli with hundreds to thousands of technical replicates across patterned surface.

In some embodiments, provided apparatus and methods assess functional responses of cells to environmental signal and/or stimuli. In some embodiments, provided apparatus and methods rely on coordination of events, for example, including: antibody secretion, virus neutralization, cell infection, and maintenance of cells during signal development.

In some embodiments, provided apparatus and methods are characterized in that they afford the ability to spatially register each individual cell pattern with each cognate well, allowing an addressable link between the contents of each nanowell and the result on the cellular behavior within each pattern to be maintained. The addressable link coupled with a high-throughput and flexible nature, makes provided apparatus and methods a highly promising tool for development of high-content screens to identify therapeutics based on function.

In some embodiments, provided apparatus and methods can be used to measure interactions between co-cultured cells in response to stimuli from micro-well, for example, target reporter cell lines can be used to measure Notch/Delta signaling between cells or intracellular signaling events in response to stimuli, such as stimulation with TNF-α.

In some embodiments, provided apparatus and methods can be used to measure/quantify interactions between cells in well and on target surface, including: secretion of antibodies with biological activity, such as: oncolytic, neutralization, enzymatic, ADCC, recruitment, bi-specific binding, antibody effector function, epitope mapping, high affinity capture, antibody mediated drug or particle delivery.

In some embodiments, provided apparatus and methods can be used to measure/quantify activation of patterned cells.

In some embodiments, provided apparatus and methods can be used to measure/quantify cytolytic activity of secreted products for killing of cells.

In some embodiments, provided apparatus and methods can be used to measure/quantify infection of patterned cells by virus produced in wells.

In some embodiments, provided apparatus and methods can be used to measure/quantify intracellular signaling of patterned cells.

In some embodiments, provided apparatus and methods can be used to measure/quantify many cell behaviors, including cell migration, proliferation, infection, motility, apoptosis, cell division, cell-cell interactions, and/or differentiation of patterned cells.

In some embodiments, provided apparatus and methods are characterized by simplicity of approach for facilitating identification of therapeutic antibodies for various applications, including those with oncolytic function, effector function, or even bi-specific binding abilities.

### EXEMPLIFICATION

### Example 1

### Assessing Viral Neutralization and/or Infection with GHOST Cell Patterning

The present Example describes use of provided cell printing technologies to assess viral infectivity on patterned GHOST cells.

According to this Example, a GHOST cell slide is prepared for printing according to the following steps (Total Time: approx. 3.5 hrs):

1) Coat one poly-lysine slide with anti-CD4 (30 μg/mL minimum) for 1 hr. Trypsinize GHOST cells. Let rest for 1 hr at 37° C. (1 hr).

2) Block CD4/poly-lysine slide and lifter-slip with 3% milk in PBS for 30 minutes. Wash 2×10 minutes in PBS; this is “CD4 slide”. (1 hr)

3) Load GHOST cells onto CD4 slide. Incubate with 3-4×10⁶ cells/120 μL GHOST cells in SF-DMEM under lifter-slip (15 min) (no polybrene). Wash slide (2×, 10 min, PBS); this is “GHOST cell slide”. (30 minutes).

4) Incubate GHOST cell slide at 37° C. in complete DMEM. Let GHOST cell slide rest for 1.5 hour. (1 hr).

Separately, virus is neutralized in wells according to the following steps (Total Time: 3 hrs):

1) Block poly-lysinecoated slide with 3% milk in PBS, then wash 2×10 min. Rinse in D1, spin dry; this is “PL slide”. (1 hr).

2) Plasma treat (3 min) and block a 250 μm stamp (1% BSA, 10 min), wash 2×10 min in PBS. (45 minutes).

3) Load 250 microwell “stamp” with virus; bring to 400 μL with HL—( 4/12 prep). Rock back and forth (6× each direction), then aspirate until wells are visible. (15 minutes); this is “virus stamp”.

4) “Print” with blocked poly-lysine slide for 2 hrs. (2 hrs).

Then, GHOST cell slide is “printed” with neutralized virus stamp according to the following steps (Total Time: 3 hrs):

1) Carefully remove PL slide from virus stamp. Replace with SF DMEM wetted GHOST cell slide. Print for 2 hrs upside-down.

2) Remove GHOST cell slide in petri dish of SF DMEM and wash 2×10 minutes in HBSS. Replace with complete media.

Printed GHOST cell slide can be imaged, for example at 48 hrs for GFP (Stain for BG and live cell). In initial experiments, it was found that infection was greater than on slides where virus was patterned, but still not as high as when slides were incubated post-cell patterning; in representative images, about 5-9 infected cells were visualized per field. Repeat without aspirating and without 2 hour neutralization was expected to give an indication of maximum infectivity that could be achieved.

### Example 2

### Assessing Viral Neutralization and/or Infection with GHOST Cell Patterning

The present Example describes use of provided cell printing technologies to assess viral infectivity on patterned GHOST cells after virus incubation with CHOb12 cells. According to this Example, a GHOST cell slide is prepared for printing (Total Time 3 5 hrs) according to the following protocol:

1) Coat two poly-lysine slides with m-anti-h-CD4 OKT 4 clone (30 μg/mL minimum) for 1 hr. Trypsinize GHOST cells. Let rest for 1 hr at 37° C. (1 hr).

2) Block CD4/poly-lysine slides and lifter-slips with 3% milk in PBS for 30 min. Wash 2×10 min. in PBS; this is “CD4 slide”. (1 hr).

3) Load GHOST cells onto CD4 slide: Rinse with D1, dry, then load cells. Incubate with 4.75×10⁶ cells/120 μL GHOST cells in SF-DMEM under lifter-slip (15 min) (no polybrene). Wash slide (2×, 5 min, PBS) (30 minutes); this is “GHOST cell slide”.

4) Incubate GHOST cell slide at 37° C. in complete DMEM. Let GHOST cell slides rest for 1.5 hours. (1.5 hr).

Separately, stamp is prepared for printing with GHOST cell slide (Total Time: 4.5 hrs) according to the following protocol:

1) Trypsinize and stain CHO-b12 (Calcein AM). Suspend to 1e6 in 2 mL of PROCHO-FBS-MSX media and stain: 30 min 1:1000 ratio at 37° C., wash and rest 30 min at 37° C. (start in parallel with step 1.1) (1.5 hrs).

2)_ Plasma treat a 250 μm (7/16, 2 hrs curing) and 100 μm (7/2, 3 hrs curing) stamp for 3 minutes. Block for 10 minutes in PBS+0 5% BSA, wash 2×10 minutes with PBS. Perform in parallel with step 1.2 and 2 1 (1 hr).

3) Load CHO b12 cells on chips. For 100 μm wells, use 500 μL of 2.5×10⁵ cells/mL to get wells with 1-6˜cells/well. For 250's, load 500 μL of 1.5×10⁵/mL to get between 6 and 10 cells per well. Wash both as needed to remove free floating cells. Image stamps with Nikon. Perform in parallel with step 4. (1 hr); this is “CHO b12 stamp”.

Then, the GHOST cell slide is printed with the CHO b12 stamp, after virus has been added (Total lime 3 hrs), according to the following protocol:

1) Load virus (4/13 HL-1 CD44 virus, 3 per chip) directly onto CHO b12 stamp, do not aspirate. Spike in 3 μg/mL of g-a-m-647 (0.3 μL). Rock back and forth several times to allow virus to mix into wells. Rinse GHOST cell coated slide with SF DMEM 2×, do not dry, then use for printing upside down for 3 hours (3 hrs).

2) Remove GHOST cell coated slide, rinse 2× with SF DMEM, then put into complete media. (0.5 hrs).

Printed slide can then, for example, be stained for CV using 0.5 μL CV/mL HBSS, in can be imaged, for example, with scope BAT gfp and CV settings, plus settings for background OR Nikon.

Initial experiments included analysis of 218 wells, having ˜50 targets cells on average (>40). When CV stain did not work efficiently, percentage-based infectivity analysis could not be made. Infected cells were counted manually after Enumerator analysis. In Enumerator, the entire block was treated as one single well. This way, infected cells were found for each block. Infected cells per each 250 μm well were then determined manually by counting the infected cells per BG stained well.

In initial experiments, for 250 μm wells, wells with three (3) CHO-b12 cell-loaded wells had enough cells attached for analysis; none of the 0-2 CHO-b12-cell-loaded wells had enough cells for analysis (these regions are usually around the edges of the stamp). In one particular experiment, only two wells with 3 CHO-b12 cells had enough cells (these are included in the infected cells plot of FIG. 17.

For 100 μm wells, cells detached after the print was removed. In some embodiments, particularly for 100 μm/50 μm well stamps, channels can be cut open to minimize damage of cell slide (i.e., GHOST cell slide in this Example) during removal of slide after print. Such opening of channels may facilitate easier release of the slide.

### Example 3

### Assessing Viral Neutralization with Inventive Cell Printing

The present Example describes use of provided microwell-assisted cell patterning (MWAP) technology to monitor viral infection of cells, and thereby to assess virus neutralization. As described, provided MWAP technology permits separate nano-scale assays to be performed in individual, discrete printed cell populations.

As described in this Example, virus (in this Example, HIV), and cells secreting virus-neutralizing antibodies were combined in individual wells of a microwell stamp that was then printed on a substrate coated with cells. As a result of the printing, that cells present at points of contact between the stamp and the substrate were mechanically disrupted, whereas cells positioned on the substrate in register with microwells remained intact. The resulting substrate therefore was characterized by spatially-patterned cells whose arrangement on the substrate reflected the structure of the microwell stamp: intact cells were present at discrete positions corresponding to wells of the stamp, and were substantially absent from discrete positions corresponding to points of contact between the stamp and the substrate. Moreover, each cluster of intact cells was exposed to the contents of a particular well in the microwell stamp. Thus, multiple individual nano-scale assays were performed simultaneously. The present Example therefore confirms that the provided technology achieves high-throughput, single-cell-scale analyses, including of complex biological systems and the interactions that occur within them.

This particular Example specifically demonstrates utility of provided technology to identify and/or characterize therapeutic antibodies for neutralization of HIV, using drastically reduced time and resources than are required for other technology formats. Those skilled in the art, reading the present disclosure, will appreciate that the described implementation can readily be adjusted to permit identification and/or characterization of other therapeutic agents (e.g., other antibodies, or nucleic acid or small molecule based therapeutics) relevant to HIV, to other infectious diseases, or to other diseases, disorders or conditions including, for example, cancer, immunological disorders, etc.

With respect to HIV, it is known that identification of high affinity anti-HIV-1 antibodies with therapeutic potential can be facilitated by the use of high-throughput antibody screens, such as plate based neutralization assays, in which culture supernatant from memory B cells is incubated with virus before infection of cells, allowing for identification of antibodies that prevent infection. Strong neutralizing antibodies, such as PG9 and PGT121 (see Walker, L. M. et al., “Broad and Potent Neutralizing Antibodies From an African Donor Reveal a New HIV-1 Vaccine Target,” 326 Science, 285-289 (2009)), have been successfully identified with these methods. However, as appreciated in accordance with the present invention, such methods are limited to plate-based formats that are time consuming, and are neither scalable nor cost effective. (See Fernandes, T. G. et al., “High-Throughput Cellular Microarray Platforms: Applications in Drug Discovery, Toxicology and Stem Cell Research,” 27 Trends in Biotechnology, 342-349 (2009)).

The present invention appreciates that improved neutralization assay formats would be valuable for a variety of reasons, including that use of neutralization assays to identify new antibodies could help identify those that neutralize using novel viral epitopes exposed during a transition state (ie: post CD4 binding), or those that are poorly represented by recombinant gp120 molecules. Zolla-Pazner, S., “Identifying Epitopes of HIV-1 That Induce Protective Antibodies,” 4 Nature Reviews Immunology, 199-210 (2004). Given the promise of neutralizing antibodies in vaccine development, and the benefit of identification of these antibodies based on function, is the present disclosure appreciates a clear need to develop functional and scalable screens for identifying potent neutralizing antibodies. van Gils, M. J. et al., “Broadly Neutralizing Antibodies Against HIV-1 Templates for a Vaccine,” 435 Virology, 46-56 (2013). As detailed in this Example, the present disclosure provides, among other things, a nanoliter-scale, function-based high-throughput screen (nano-neutralization assay) for the detection of HIV infection of cells, and the identification and/or characterization of cells secreting anti-HIV-1 antibodies. Provided methods and apparatus can drastically reduce the time and resources required to discover antibodies with high therapeutic potential.

### Materials & Methods

Tissue Culture

The following reagent was obtained through the AIDS Reagent Program, Division of AIDS, NIAID, NIH: Ghost(3)X4/R5 from Drs. Vineet N. KewalRamani and Dan R. Littman. GHOST cells were maintained in complete media (Dulbecco's Modified Eagle Medium (supplier) with 10% Fetal Bovine Serum (FBS), supplemented with 1% Penicillin/Streptomycin, 500 μg/ml G418 (supplier), 100 μg/ml hygromycin (supplier), and 1 μg/ml puromycin (supplier)) and discarded after 15 passages. Chinese hamster ovary (CHO) cell lines producing b12 (anti-gp120), 2F5 (anti-gp41) or b6 antibodies (courtesy of D. Burton, Scripps Institute) were cultured in ProCHO-5 media (Lonza) with 10% FBS, 1× HT supplement (Gibco), 1× GS supplement (Sigma-Aldrich), 100 U/mL penicillin, 100 mg/mL streptomycin and 50 mM 1-methionine sulfoxime (Sigma-Aldrich). A human B cell hybridoma cell line producing the 4D20 (anti-hemagglutinin) antibody (courtesy of J. Crow, Vanderbilt University) was adapted to grow in HL-1 medial containing 15% (v/v) FBS, 2 mM 1-glutamine, and 1 mM sodium hyruvate. Cultures were passaged every 3-5 days and using when 60-70% confluent. ROCK inhibitor (Y-27632) and FAK inhibitor (PF-573228) were purchased from Tocris Bioscience, and suspended according to the manufactures instructions.

Fabrication of Nanowell Arrays

Nanowell arrays with the indicated geometries were manufactured as previously described using poly(dimethylsiloxane) (Sylgard 184). (See Han, K. et al., “Parallel Measurement of Dynamic Changes in Translation Rates in Single Cells,” 11 Nat Meth, 86-93 (2013) the contents which is incorporated herein by reference in its entirety. After curing in custom-made injection molds, arrays were removed, covered and stored at ambient conditions until use. Arrays were treated with oxygen plasma (PDC-001, Har-rick Plasma) for 4 minutes immediately prior to use, then placed directly into 0.5% (v/w) Bovine Serum Albumin (BSA) in Phosphate Buffered Saline (PBS).

Cell Patterning

GHOST cells were washed in complete media and resuspended in serum-free DMEM (SF-DMEM) at 1.5×10⁶ cells per 120 μl unless indicated otherwise. Poly-l-lysine slides were coated with 85 μl of 25 μg/ml mouse anti-human CD4 antibody diluted in borate buffer under a LifterSlip™ (EMS). The slides were blocked for 30 min in 3% (v/w) milk in PBS and washed twice for 5 min in PBS on a rocker. 120 μl of GHOST cell suspension was then added to each slide under a LifterSlip™ (EMS) and incubated for 15 min at room temperature (RT). The cell-coated slides were then incubated in complete GHOST cell media for 1 hr at 37° C. for attachment to the surface. For patterning, 350 μl of 5 ug/ml fluorescently labeled goat anti-mouse IgG (H+L) (Invitrogen) diluted in SF-DMEM was then added to each nanowell device to cover the entire surface. The cell-coated slide was then brought in contact with the nanowell device to seal the wells, and the assembly was incubated for 2-3 hrs at 37° C. After removal, the cell-patterned slides were washed twice for 5 min in PBS to wash away disrupted cells. Before imaging, slides were fixed to a 4-well dish with 2% agarose (Difco Agar Noble, BD) at the four corners of the slide. For viability staining, 4 ml of 2 μM calcein violet AM (Invitrogen) diluted in PBS was added to each slide immediately prior to imaging.

Imaging and Data Analysis

The cell arrays were imaged with a Zeiss epi-fluorescence microscope. The slides were aligned using the background channel and imaged in an automated fashion. The images were analyzed with a custom analysis software, which uses the background fluorescence channel to locate each element of the array within the image, identifies the cells that are fluorescent in any channel of interest, and returns the number of cells per array element, and their intensity, size, and position

Pseudovirus Production

Pseudovirus was produced using transient transfection of HEK 293 cells as previously described, with modifications (Greene et al.). Briefly, HEK 293 cells were seeded into T25 tissue culture flasks and allowed to adhere overnight. The following morning, 5 ug each of pCMV4-BlaM-Vpr, pSG2Δenv, and pYU2ΔCT plasmids was transfected into the cells using Genjet (Signagen, Rockville, Md.) according to manufacturer's protocol in serum free HL-1 media. After 48 hrs, media was collected and virus spun concentrated 10-fold using Amicon Ultra 100K centrifugal filter units (Millipore, Bellerica, Mass.) and stored at −80° C. until use.

Microengraving

Microengraving to detect antibody titres secreted from CHO b12 cell line was performed as previously described. Poly-l-lysine slides were coated with Zymax goat-anti-human (25 μg/mL, Life Technologies) capture antibody. Microengraving was allowed to proceed for three hours, after which, protein arrays were scanned on a GenePix 4200AL and analyzed in GenePix Pro 6.0 (Molecular Devices). Data analysis was performed as previously described (Ogunniyi, 2014).

Virus Infectivity Assay

GHOST cells were seeded at 50,000 cells per well in a 24-well plate (BD Falcon) and allowed to adhere overnight. Serial dilutions of psuedovirus were added to each well the following day for infection. After three hours, cells were washed and placed back in the incubator. After 48 hours, cells were detached with trypsin, fixed with paraformaldehyde, and infection assayed by flow cytometry.

Virus infection was also assessed using the nanowells. Nanowells were loaded with 500 μL of virus then sealed with a cell-coated glass slide as described above. After 3 hrs, arrays were removed, washed 2× with PBS, and placed into culture media for 48 hrs. Cell-arrays were scanned as described above to determine the number of infected cells per pattern.

Cell Migration Inhibition Assay

Cell patterns were formed as described above. Arrays were imaged at 0, 24, and 48 hrs immediately after addition of 1 μM calcein AM live cell stain (Life Technologies) in PBS. Media additives were diluted directly into culture media at the indicated concentration. Cell proliferation (P) was calculated per block (4 wells per block) as the ratio of cells at time of imaging to the number of starting cells at time zero. Cell migration (M) was calculated as N_{full}×P−N_{well}, where N_{full} is the number of cells per field of view, P is the proliferation rate, and N_{well} is the number of cells per well.

Neutralization Assay

Cell patterns were formed as described above with the following modifications. Antibody secreting cell lines were stained with 1 uM CellTraker™ red and 1 uM sytox green (Life Technologies). Arrays were loaded to a density of between 0 and 10 cells per well, and transmitted and fluorescent images were acquired with an automated epifluorescence microscope (Carl Zeiss) fit with an EM-CCD camera (Hamamatsu). The array containing antibody secreting cells was loaded with 400 μL pseudovirus containing 4 μg/mL of goat-anti-mouse background stain ( ), then brought into contact with a GHOST cell coated slide for 3 hrs. The GHOST cell coated slide was then removed, washed 2× with PBS, and placed into media for 24 hrs. At 24 hrs, media was replaced with fresh media containing 10 μM FAK inhibitor (PF-573228). Cell arrays were imaged at 48 hrs immediately after addition of 2 μM calcein violet AM and 3 μg/mL Hoest nuclear stain.

Statistical Analysis

All statistical analysis was performed using Prism 6 (V 6.0 e). Statistical tests used are indicated where appropriate.

### OTHER EMBODIMENTS AND EQUIVALENTS

While the present disclosure has explicitly discussed certain particular embodiments and examples of the present invention, those skilled in the art will appreciate that the invention is not intended to be limited to such embodiments or examples. On the contrary, the present invention encompasses various alternatives, modifications, and equivalents of such particular embodiments and/or example, as will be appreciated by those of skill in the art.

To give but a few examples, methods and diagrams of should not be read as limited to a particular described order or arrangement of steps or elements unless explicitly stated or clearly required from context (e.g., otherwise inoperable). Furthermore, different features of particular elements that may be exemplified in different embodiments may be combined with one another in some embodiments.

1. A patterned cell array comprising:
a substrate having a surface; and
intact cells associated with the surface in a pattern that is spatially-registered relative to a microwell device in that intact cells are present on the surface in discrete locations (“cell locations”) corresponding to wells in the microwell device, and are substantially absent from the surface in discrete locations (“cell-free locations”) corresponding to inter-well regions that represent points of contact between the substrate surface and the microwell device if they are pressed together.

2. The patterned cell array of claim 1, wherein the surface of the substrate is functionalized with an adhesion agent.

2A. The patterned cell array of claim 2, wherein the adhesion agent is characterized in that it interacts specifically with cells of a particular cell type as compared with cells of one or more different cell types.

2B. The patterned cell array of claim 2, wherein the adhesion agent is or comprises an adhesion molecule.

2C. The patterned cell array of claim 2, wherein the adhesion agent is or comprises an antibody to a cell surface marker (e.g. α-CD4).

3. The patterned cell array of claim 2, wherein the substrate surface is uniformly coated with the adhesion agent.

4. The patterned cell array of claim 1, wherein cells in one or more of the cell locations are present in a monolayer.

5. The patterned cell array of claim 1, wherein the substrate is formed of or comprises glass.

5A. The patterned cell array of claim 1, wherein the substrate is or comprises a standard microscope slide.

6. An apparatus comprising:
a substrate having a surface on which living cells have been substantially uniformly disposed; and
a microwell device in contact with the surface so that cells on the surface are disrupted at points of contact between the microwell device and the surface, and cells on the surface are substantially intact at locations corresponding to the wells of the microwell device.

6A. The apparatus of claim 6, wherein one or more of the microwells contains a cytotoxic agent.

6B. The apparatus of claim 6, wherein one or more of the microwells contains an antibody secreting cell.

6C. The apparatus of claim 6, wherein one or more of the microwells contains a virus.

6D. The apparatus of claim 6, wherein one or more of the microwells contains growth factor.

6E. The apparatus of claim 6, wherein one or more of the microwells contains co-cultures of cells.

6G. The apparatus of claim 1, wherein at least one well of the array of wells comprises at least one antibody-producing cell.

7. The apparatus of claim 6, wherein at least one well of the array of wells further comprises a virus and the at least one antibody-producing cell forms a neutralizing antibody specific to the virus.

8. The apparatus of claim 2, at least one well of the array of wells comprises fluorescently-labeled antibodies specific to antibodies functionalized to the surface of the substrate.

9. The apparatus of claim 1, wherein each well has a diameter of 10 to 250 microns.

10. The apparatus of claim 1, wherein the substrate is glass.

11. The apparatus of claim 1, wherein the slab is moldable.

12. The apparatus of claim 1, wherein the slab is etched.

13. The apparatus of claim 1, wherein the slab is deposited.

14. The apparatus of claim 1, wherein the slab is made of poly(dimethylsiloxane) (PDMS).

15. The apparatus of claim 1, wherein the reversible seal forms a substantially fluid tight seal between the substrate and the slab.

16. The apparatus of claim 1, wherein the substrate comprises a plastic.

17. The apparatus of claim 1, wherein the substrate is substantially planar.

18. The apparatus of claim 1, wherein the surface of the slab is substantially planar.

19. A substrate with a surface coated with a layer of cells, wherein the surface is characterized in that at least one cell is disrupted when the substrate surface is reversibly contacted to a slab forming a pattern defined by an array of wells formed in a surface of the slab.

20. A method comprising steps of:
providing a substrate having a surface on which living cells are substantially uniformly distributed;
providing a slab comprising an array of wells formed into a surface of the slab;
sealing the substrate against the surface of the slab so that cells on the substrate surface are mechanically disrupted at points of contact between the substrate surface and the slab surface, and cells on the substrate remain intact at positions corresponding to wells in the slab, so that a patterned array of cells that is spatially registered relative to the wells is generated on the substrate.

21. The method of claim 20, the step of releasing the substrate with the stimulated cells from the slab, further comprising washing away disrupted cells.

21A. The method of claim 20, wherein, wherein each well is defined by a volume that is sized to contain about a nanoliter of liquid.

21B. The method of claim 20, wherein at least one well contains an environmental stimulus.

22. The method of claim 20, wherein the step of providing a substrate, comprises providing a substrate that is glass.

23. The method of claim 20, wherein the step of providing a substrate comprises providing a substrate formed from a plastic material.

24. The method of claim 20, the step of providing a substrate, wherein the substrate is substantially planar.

25. The method of claim 20, the step of providing a substrate, wherein the slab is made of poly(dimethylsiloxane).

26. The method of claim 20, the step of providing a slab, wherein the slab is substantially planar.

27. The method of claim 20, the step of incubating the cells in the wells, wherein the of cells to remain in contact with well contents for a desired period.

28. The method of claim 20, wherein the steps of sealing and incubating are performed in less than about 12 hours.

29. The method of claim 20, wherein the step of sealing forms a substantially fluid tight seal between the slab and the substrate.

30. The method of claim 20, further comprising incubating the cells in the wells.

31. The method of claim 20, further comprising releasing the substrate that has the stimulated cells from the slab.

32. The method of claim 20, further comprising one or more steps of:
measuring and/or quantifying activation of patterned cells;
measuring and/or quantifying cytolytic activity;
measure/quantify cytolytic activity of secreted products for killing of cells;
measuring and/or quantifying secretion of cytotoxic proteins by cells;
measuring and/or quantifying active killing of patterned cells;
measuring and/or quantifying antibody mediated cellular cytotoxicity;
measuring and/or quantifying infection of patterned cells by viral particles produced in wells;
measuring and/or quantifying antibody secretion;
measuring and/or quantifying virus neutralization;
measuring and/or quantifying cell infection;
measuring and/or quantifying interactions between co-cultured cells in response to stimuli;
measuring and/or quantifying secretion of antibodies with biological activity;
measuring and/or quantifying infection of patterned cells by virus produced in wells;
measuring and/or quantifying intracellular signaling of patterned cells;
measuring and/or quantifying cell migration;
measuring and/or quantifying cell proliferation;
measuring and/or quantifying cell infection;
measuring and/or quantifying cell motility;
measuring and/or quantifying cell apoptosis;
measuring and/or quantifying cell division;
measuring and/or quantifying cell-cell interactions; and/or measuring and/or quantifying differentiation of patterned cells.
